(19) United States
(12) Patent Application Publication
(10) Pub. No.: US 2023/0293602 A1
(43) Pub. Date: Sep. 21, 2023

(54) COMBINATION IMMUNOTHERAPY METHODS FOR THE TREATMENT OF CANCER

(71) Applicant: The Board of Regents of The University of Texas System, Austin, TX (US)

(72) Inventors: Andrew Young KOH, Southlake, TX (US); Yongbin CHOI, Dallas, TX (US)

(21) Appl. No.: 18/042,231

(22) PCT Filed: Aug. 20, 2021
(86) PCT No.: PCT/US2021/047001

§ 371 Date: Feb. 20, 2023

Related U.S. Application Data
(60) Provisional application No. 63/068,127, filed on Aug. 20, 2020.

(51) Int. Cl. A61K 35/742; A61P 35/00

(52) U.S. Cl. CPC A61K 35/742; A61P 35/00

(57) ABSTRACT

Methods and compositions for the treatment of cancer are provided. The compositions comprise gut bacterial lysates and/or components thereof. The method comprises administration to a subject in need thereof of a gut bacterial lysate. Also provided are methods comprising administration of a gut bacterial lysate in combination with another cancer treatment.

### CROSS-REFERENCE TO RELATED APPLICATION

The present application claims priority to U.S. Provisional Patent Application No. 63/068,127, filed on Aug. 20, 2020, entitled “COMBINATION IMMUNOTHERAPY METHODS FOR THE TREATMENT OF CANCER,” which is specifically incorporated by reference in its entirety herein.

### ACKNOWLEDGEMENT OF GOVERNMENT SUPPORT

This invention was made with government support under Grant No. CA231303 awarded by the National Institutes of Health. The government has certain rights in this invention.

### BACKGROUND

### 1. Field

The present inventive concept is directed to methods and compositions for the treatment of cancer, that include administration of a gut bacterial lysate. The present inventive concept is also directed to methods and compositions for the treatment of cancer that include administration of a gut bacterial lysate in combination with another cancer treatment.

### 2. Discussion of Related Art

Standard cancer treatments such as chemotherapy, radiation, and immunotherapy can help patients achieve durable remissions. However, a substantial number of patients fail to benefit from one or more of these therapies, and others experience severe reactions to them. For example, in the case of immune checkpoint inhibitor therapy (ICT), a form of immunotherapy, severe autoimmune adverse events can include dermatitis, colitis, hepatitis, and hypophysitis. Adverse effects associated with another type of immunotherapy, CAR-T cells, include cytokine release syndrome and neurotoxicity. Consequently, there is a need for both improved cancer therapies and methods to make current therapies more tolerable and efficacious.

There is mounting evidence that gastrointestinal tract bacteria, collectively known as the gut microbiota, can influence and modulate host immune responses and/or augment cancer therapy. For instance, in preclinical mouse models, the composition of the host gut microbiota is a major factor determining immune checkpoint inhibitor therapy (ICT) response. Additionally, germ-free or antibiotic-treated tumor-bearing mice exhibit significantly diminished responses to immune therapy. B16 melanoma-bearing mice treated with Bifidobacterium spp. show increased tumor DC antitumor immune gene expression and enhanced anti-PD-L1 immunotherapy response. Furthermore, B. thetaiotaomicron (B. thetaiotaomicron or B. theta) or B. fragilis may be important for anti-CTLA4 antibody anti-B16 melanoma in vivo efficacy. Dendritic cells (DCs) and T cells coincubated with either of these Bacteroides species in vitro increased T-cell interferon γ production and in vivo tumor growth inhibition. In all the above studies, the gut bacteria induced maturation of anti-melanoma DCs and T cells.

Attempts have been made to use bacterial products and ligands to bolster host immune anti-cancer effects. Most notably Coley's toxin, a mixture of heat-killed pathogenic Gram-negative bacteria (Serratia marcescens) and pathogenic Gram-positive bacteria (Streptococcus pyogenes), had been used from the 1890s to 1950s as cancer therapy. Coley's toxin was typically directly injected into the tumor. Unfortunately, injection of pathogenic bacterial constituents (e.g., lipopolysaccharide, LPS) can induce sepsis and lead to death. Oral administration of probiotics and fecal transplants to introduce gut bacteria into subjects are plagued by difficulties in consistently and permanently engrafting in the gut of the subject. Such techniques often require patients to receive antibiotic treatments prior to introduction of gut microbiota to facilitate colonization of the gut microbiota of interest. Additionally, fecal transplants have sometimes resulted in fatal infections attributable to the fecal transplant itself. Accordingly, additional methods and compositions for increasing the effectiveness of immunotherapy treatments are desirable. Further, additional methods and compositions for bolstering subject's immune anti-cancer effects are desirable.

### BRIEF SUMMARY

The following brief description is provided to indicate the nature of the subject matter disclosed herein. While certain aspects of the present inventive concept are described below, the summary is not intended to limit the scope of the present inventive concept.

In various aspects, a pharmaceutical composition is provided herein, the composition comprising one or more bacterial lysates from one or more species of a Gram-positive bacterial cell; one or more bacterial lysates from one or more species of Gram-negative bacterial cell; and at least one pharmaceutically acceptable carrier and/or excipient,

In further aspects, another pharmaceutical composition is provided, the composition comprising: one or more components from one or more bacterial lysates from one or more species of a Gram-positive bacterial cell; one or more components from one or more bacterial lysates from one or more species of a Gram-negative bacterial cell; and at least one pharmaceutically acceptable carrier and/or excipient. In various aspects, in any of the pharmaceutical compositions herein, the one or more species of a Gram-positive bacterial cell is F. prausnitzii, F. johnsonii, E. faecalis, Enterococcus sp., E. faecium, E. gallinarum, E. hirae, B. producta, C. bolteae, B. pseudolongum, L. acidophilus, or any combination thereof and the Gram-negative bacterial lysate comprises lysate from B. thetaiotaomicron, B. vulgatus, B. ovatus, B. uniformus, P. copri, or A. muciniphila.

In any pharmaceutical composition herein, the one or more species of a Gram-negative bacterial cell is B. thetaiotaomicron, B. vulgatus, or any combination thereof.

In any pharmaceutical composition herein, the one or more species of a Gram-positive bacterial cell is E. faecium, E. faecalis, E. gaffinarum, E. hirae, or any combination thereof.

In any pharmaceutical composition herein, the composition may comprise one or more bacterial lysate from one or more species of bacteria having genomic DNA with a CpG abundance substantially similar to a CpG abundance in genomic DNA of a gut bacterium and a pharmaceutically acceptable carrier and/or excipient. In various embodiments, the one or more species of bacteria having genomic DNA with a CpG abundance substantially similar to a CpG abundance in genomic DNA of a gut bacterium is F. prausnitzi, B. thetaiotaomicron, B. producta, B. vulgatus, or any combination thereof. In further embodiments, the one or more gut bacterium is F. prausnitzi, B. thetaiotaomicron, B. producta, B. vulgatus, or any combination thereof.

In any pharmaceutical composition herein, the composition may comprise one or more bacterial lysate from a species of bacteria that comprises a Lipid A structure substantially similar to a Lipid A in B. thetaiotaomicron and at least one pharmaceutically acceptable carrier and/or excipient. In various embodiments, the Lipid A structure comprises a monophosphoryl Lipid A comprising 5-6 acyl chains.

In still further aspects, another pharmaceutical composition is provided, the composition comprising one or more bacterial lysates from one or more species of a Gram-positive bacterial cell; one or more bacterial lysates from one or more species of a Gram-negative bacterial cell; and a pharmaceutically acceptable carrier and/or excipient, wherein the one or more species of Gram-negative bacterial cell comprises a monophosphoryl Lipid A comprising 5-6 acyl chains such that Gram-negative bacterial lysate comprises the same, and wherein the one or more Gram-positive bacterial lysates and/or the one or more Gram-negative bacterial lysates comprise genomic DNA with a CpG abundance substantially similar to that of a CpG abundance in genomic DNA of a gut bacterium.

The one or more species of Gram-positive bacterial cells may comprise lipoteichoic acid (LTA) having a structure substantially similar to the LTA found in F. prausnitzii. In various embodiments, the one or more species of Gram-positive bacterial cell is F. prausnitzii.

In some embodiments, the one or more Gram-positive bacterial lysates and one or more Gram-negative bacterial lysates collectively contain ligands that are capable of binding to toll-like receptor 2, toll-like receptor 4, and NOD2 on a target cell, and such binding being sufficient to activate a cellular response in such target cell.

In various embodiments, the composition may be formulated as a liquid formulation and the pharmaceutically acceptable carrier and/or excipient comprises a phosphate buffered saline solution. In various embodiments, the liquid formulation comprises a pH of from about 6.8 to 7.5. In some embodiments, the liquid formulation comprises a pH of from about 7.35 to about 7.45.

In various aspects, a use of any pharmaceutical composition provided herein for the treatment of cancer in a subject is provided.

In various aspects, a method is provided for the treatment of cancer in a subject in need thereof, the method comprising: (a) administering a therapeutically effective amount of any pharmaceutical composition described herein to the subject. In various aspects, the method may further comprise (b) administering a therapeutically effective amount of a cancer treatment to the subject. In various embodiments, steps (a) and (b) are administered at least partially simultaneously.

In the methods provided herein, step (a) may comprise orally administering the pharmaceutical composition to the subject. In other embodiments, step (a) comprises parenterally administering the pharmaceutical composition to the subject. The parenteral administration of the pharmaceutical composition in step (a) may be intravenous, intraperitoneal, intramuscular, intrathecal, or subcutaneous. For example, the parenteral administration of the pharmaceutical composition in step (a) can be subcutaneous.

In some embodiments, step (a) comprises subcutaneously administering the pharmaceutical composition ipsilaterally to a tumor in the subject. In some embodiments, step (a) comprises subcutaneously administering the pharmaceutical composition contralaterally to a tumor in the subject.

In some embodiments, the parenteral administration of the pharmaceutical composition in step (a) is intravenous.

In some embodiments, step (a) comprises administering the pharmaceutical composition using at least two administration techniques selected from the group consisting of subcutaneous, intravenous, intraperitoneal and oral administration. For example, in some embodiments, step (a) comprises administering the pharmaceutical composition both intravenously and subcutaneously close to a draining lymph node of a metastasis.

In any of the methods provided herein, the cancer treatment in step (b) may be an immunotherapy treatment.

In various embodiments, the cancer immunotherapy treatment may comprise administering to the subject an immune checkpoint inhibitor (ICT), modified immune cells, a bispecific antibody, or any combination thereof.

In various embodiments, the cancer immunotherapy treatment comprises administering an immune checkpoint inhibitor (ICT) and the immune checkpoint inhibitor (ICT) comprises an anti-PD-1 therapy, an anti-PD-L1 therapy, an anti-CTLA-4 therapy or any combination thereof.

In some embodiments, the anti-PD-1 therapy comprises pembrolizumab, nivolumab, cemiplimab, spartalizumab, sintilimab, tislelizumab, toripalimab, dostalimab or combinations thereof; the anti-PD-L1 therapy comprises atezolizumab, avelumab, durvalumab KN035, AUNP12, or combinations thereof, and/or the anti-CTLA-4 therapy comprises ipilimumab.

In some embodiments, the cancer immunotherapy treatment comprises administering (a) an anti-PD-L1 or an anti-PD-1 therapy; and (b) an anti-CLTA-4 therapy.

In still further embodiments, the method comprises administering a composition comprising one or more bacterial lysates from F. prausnitzii, B. thetaiotaomicron, or any combination thereof.

In various aspects, the cancer immunotherapy treatment comprises administering modified immune cells to the subject and the modified immune cell comprises a modified natural killer (NK) cell, a modified dendritic cell (DC), a CAR-T cell or any combination thereof.

In various embodiments, the cancer immunotherapy treatment comprises administering a bispecific antibody to the subject.

In any of the methods of treating cancer provided herein, the cancer may be selected from the group consisting of squamous cell head and neck cancer, colon cancer, colorectal cancer, Acute myeloid leukemia (AML), Chronic myeloid leukemia (CML) Acute lymphoblastic leukemia (ALL), Merkel cell carcinoma, cutaneous squamous cell carcinoma, hepatocellular carcinoma, advanced renal cell carcinoma, microsatellite instability-high (MSI-H) or mismatch repair deficient (dMMR) cancers, cervical cancer, small cell lung cancer, non-small cell lung cancer, triple-negative breast cancer, gastric and gastroesophageal junction (GEJ) carcinoma, classical Hodgkin lymphoma, primary mediastinal B-cell lymphoma (PMBCL), and locally advanced or metastatic urothelial cancer. For example, in some embodiments, the cancer is colon cancer or colorectal cancer. In some embodiments, the cancer is melanoma. In some embodiments, the cancer is metastatic melanoma. In some embodiments, the cancer is acute B-cell lymphoblastic leukemia. In some embodiments, the cancer is non-small cell lung cancer.

In any of the methods provided herein, step (a) may comprise administering from about 0.0005 mg/kg to about 10 mg/kg of the pharmaceutical composition to the subject. For example, in some embodiments, step (a) comprises administering from about 0.3 to 9.8 mg/kg of the pharmaceutical composition to the subject.

In various aspects, a kit for use in the treatment of cancer in a subject in need thereof is provided, the kit comprising: (i) one or more gut bacterial lysates in a composition formulated for oral or parenteral administration; and (ii) a cancer immunotherapy treatment comprising: (a) one or more compositions suitable for use in immune checkpoint inhibitor therapy (ICT); b) one or more compositions suitable for immune cell transfer therapy; or (c) a bispecific antibody.

In various aspects, the one or more gut bacterial lysates in a composition formulated for oral or parenteral administration is a pharmaceutical composition provided herein.

In various embodiments, the one or more compositions suitable for use in immune checkpoint inhibitor therapy (ICT) is selected from the group consisting of ipilimumab, pembrolizumab, nivolumab, cemiplimab, spartalizumab, sintilimab, tislelizumab, toripalimab, dostalimab, atezolizumab, avelumab, durvalumab, KN035, AUNP12 and any combination thereof.

In various embodiments, the one or more compositions suitable for immune cell transfer therapy may comprise a modified immune cell selected from: a modified NK cell, a CAR-T cell, or any combination thereof

### BRIEF DESCRIPTION OF THE DRAWINGS

The description will be more fully understood with reference to the following figures and data graphs, which are presented as various embodiments of the present inventive concept and should not be construed as a complete recitation of the scope of the present inventive concept, wherein:

FIG. 1 depicts an experimental protocol for determining the effect of administration of gut microbiota lysates in combination with ICT therapy in a mouse melanoma model.

FIG. 2 depicts data indicating that administration of gut microbiota lysates compensate for antibiotic-induced inhibition of ICT efficacy in mice with melanoma.

FIG. 3 depicts data demonstrating the effect of administration of gut microbiota lysates in combination with ICT therapy on percent survival of mice with melanoma.

FIG. 4 depicts comparison data showing that Coley's Toxin induces greater mortality than administration of gut microbiota lysates in healthy wild type mice.

FIG. 5 is a diagram of an experimental protocol to collect samples and test for enriched populations of bacteria in patients undergoing CAR-T therapy.

FIG. 6 shows commensal bacteria populations that are enriched in patients having B-cell recovery following CAR-T therapy (e.g., failure) or no B-cell recovery following CAR-T therapy (success).

FIG. 7 shows enriched gut microbial species in patients with positive (left) or negative (right) response to CAR-T therapy.

FIG. 8 is a diagram that shows an exemplary procedure to isolate cell lysis from the indicated bacteria.

FIGS. 9A and 9B show CD40+ and CD80+ expression, respectively, in dendritic cells (CD11c+) following stimulation with lysates of the indicated bacteria.

FIG. 10 is a diagram that shows an experiment to test the effect of activated dendritic cells on CAR-T cells in the presence of tumor cells.

FIG. 11 is a bar graph showing the number of viable CAR-T cells observed after treatment with indicated bacterial lysate alone or with dendrite cells and control or CAR-T cells.

FIG. 12 is a bar graph showing IFN-γ levels in T cells in samples comprising activated dendritic cells, cell lysates and CAR-T immunotherapy.

FIG. 13 shows gut microbiome profiles of C57BL/6 mice from Jackson and Taconic determined by 16S rRNA sequencing (V4 region) from gDNA extracted from fecal specimens collected from these mice.

FIGS. 14A and 14B show an experimental protocol and results thereof to measure tumor volume in mice treated with anti-PD-1 therapy with and without antibiotics and/or Bt/Fp lysate.

FIGS. 15A and 15B show an experimental protocol and corresponding plot of tumor volume over time in mice with colorectal cancer (MC38) receiving intratumoral injections of PBS or Bt/Fp microbiota lysate (BFML IT).

FIGS. 15C and 15D show an experimental protocol and corresponding plot of tumor volume in mice with colorectal cancer (MC38) receiving subcutaneous injections of an anti-PD-1 antibody or Bt/Fp microbiota lysate (BFML SQ, ipsilateral side of tumor).

FIG. 16 is a survival curve showing animal survival up to 26 days after tumor inoculation and treatment with antibiotics, ICT and/or Bt/Fp lysate. Animals tested were either WT or KO for TLR2 and TLR4

FIG. 17 is a survival curve showing animal survival out to 38 days after tumor inoculation in WT mice treated with ICT and Fp lysate alone, Bt lysate alone or Fp and Bt lysate together. All lysates were administered subcutaneously.

FIGS. 18A and 18B show (A) an experimental protocol where Jackson C57BL/6 mice were treated with antibiotics, injected with B16-F10 and treated with BFML or live Bt/Fp via oral gavage and (B) a survival curve of various treatment groups.

FIGS. 19A and 19B show CD40+ (18A) and CD80+ (18B) expression in dendritic cells after contact with lysates prepared from the indicated bacteria.

FIG. 20 shows a structural image of Lipid A isolated from Enterobacteriaceae and Bacteroides strains (left) and a plot of CD40+ expression in dendritic cells contacted with indicated types and quantities of bacterial cell lysate (right). Sm (Serratia marcescens) and Sp (Streptococcus pyogenes) are components found in Coley's toxin.

FIG. 21 is a MALDI-TOF MS plot analyzing Lipid A isolated from the indicated bacteria.

FIG. 22 is a diagram of the interaction of CpG DNA in immune systems.

FIG. 23 is a diagram showing relative abundance of CpG motifs across different bacterial species using data obtained from Kant et al., J. Medical Microbiology 2014.

FIGS. 24A and 24B are scatter plots showing (A) CpG abundance in different commensal (non-pathogenic) or pathogenic Gram-negative populations and (B) CpG abundance in select Gram-negative gut microbiota candidates.

FIGS. 24C and 24D are scatter plots showing (C) CpG abundance in different commensal (non-pathogenic) or pathogenic Gram-positive populations and (D) CpG abundance in select Gram-positive gut microbiota candidates.

FIG. 25 is a plot showing average CD40+ expression detected in dendritic cells stimulated with indicated lysates of bacteria. Sm/Sp refers to Coley's toxin.

FIGS. 26A, 26B and 26C show (A) an experimental schema describing ethanol extraction of aqueous (polar) and organic (nonpolar) phases of Bt/Fp lysates and corresponding activation of dendritic cells treated with whole lysate, or each phase separately (measured by CD40 (FIG. 26B) and CD80 (FIG. 26C) expression).

FIGS. 27A and 27B show percentage of CD40+ and CD80+ cells, respectively, following treatment with indicated bacterial lysates or fractions thereof that had been treated or untreated with DNase.

FIG. 28 show percentage of CD40+ dendritic cells (CD11c+) treated with vehicle, extracted genomic DNA (gDNA) from B. thetaiotaomicron, or CpG alone.

FIGS. 29A and 29B show percentage of CD40+ and CD80+ dendritic cells, respectively, following treatment with normal Bt/Fp lysate or lysate after physical denaturation (60-minute boiling) or chemical denaturing (treatment with protease from Streptomyces griseus).

FIG. 30 shows gut microbiota lysates agonizing different mouse pattern recognition receptors (TLR2, TLR4, TLR5 and NOD2), as measured by immunoassays.

FIGS. 31A and 31B show (A) an experimental schema to test effect of alternative lysates on immunotherapy in mice and (B) tumor volume in mice in different treatment groups.

### DETAILED DESCRIPTION

The present disclosure provides compositions and methods for the treatment of cancer in a subject in need thereof. The disclosure is based, at least in part, on the surprising discovery that lysates from certain bacteria can be used to treat various cancers and can be used to augment and improve other cancer therapies. It was further surprising that the use of lysates from certain gut bacteria for the treatment of cancer, retain efficacy without inducing sepsis (unlike Coley's toxin which does induce sepsis). As described in more detail herein, various combinations of bacterial lysates can improve the outcome for various cancer therapies. The presently disclosed compositions and methods utilize lysates of dead gut microbiota and therefore have advantages over techniques that use live cells which are often accompanied by potentially adverse effects with introducing new bacteria into the subject. Additionally, the difficulties associated with the need for causing new live bacteria to colonize and take hold in the gut of a subject are avoided by the presently disclosed compositions and techniques.

For the purposes of promoting an understanding of the principles of the present disclosure, reference will now be made to embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

The terminology used herein is for the purpose of describing particular embodiments only and is not intended to be limiting of the invention.

As used herein, the terms “comprise”, “comprising”, “include”, “including”, “have”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the term “method” or “methods” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Throughout this application, various embodiments may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

### 1. Compositions

In various aspects, compositions provided herein comprise one or more gut bacterial lysates, one or more components from a gut bacterial lysate, or one or more synthetic analogues of one or more components of the gut bacterial lysate. The bacterial lysates may be from one or more species of bacterial cells. In various embodiments, the compositions are pharmaceutical compositions that comprise at least one pharmaceutically acceptable carrier and/or excipient.

As used herein, the term “lysate” refers to the collective components of lysed cells where no particular component has been intentionally purified for or intentionally removed (intentionally removed does not denote components that may be unintentionally lost through standard lysis processes). The term “lysate” should not be understood to include a purified component of a cell, a live intact cell, or a dead intact cell. The term “components from a gut bacterial lysate”, “components from a bacterial lysate”, “components from a lysate”, “components from a Gram-positive bacterial lysate”, “components from a Gram-negative bacterial lysate”, or similar, refer to one or more components that have been intentionally purified or intentionally removed from a bacterial lysate or a bacterial lysate where one or more components have been intentionally removed from the bacterial lysate.

In various embodiments, the composition may comprise one or more bacterial lysates from one or more species of a Gram-positive bacterial cell and one or more bacterial lysates from one or more species of Gram-negative bacterial cell. In some embodiments, the composition is a pharmaceutical composition and the composition comprises one or more bacterial lysates from one or more species of a Gram-positive bacterial cell; one or more bacterial lysates from one or more species of Gram-negative bacterial cell; and at least one pharmaceutically acceptable carrier and/or excipient.

In some embodiments, the composition may comprise one or more components from one or more bacterial lysates from one or more species of a Gram-positive bacterial cell and one or more components from one or more bacterial lysates from one or more species of a Gram-negative bacterial cell. In some embodiments, the composition is a pharmaceutical composition and the composition comprises one or more components from one or more bacterial lysates from one or more species of a Gram-positive bacterial cell; one or more components from one or more bacterial lysates from one or more species of a Gram-negative bacterial cell; and at least one pharmaceutically acceptable carrier and/or excipient.

In some embodiments, the composition may comprise one or more synthetic analogues of one or more components from one or more bacterial lysates from one or more species of a Gram-positive bacterial cell and one or more synthetic analogues of one or more components from one or more bacterial lysates from one or more species of a Gram-negative bacterial cell lysate. In some embodiments, the composition is a pharmaceutical composition and the composition comprises one or more synthetic analogues of one or more components from one or more bacterial lysates from one or more species of a Gram-positive bacterial cell; one or more synthetic analogues of one or more components from one or more bacterial lysates from one or more species of a Gram-negative bacterial cell lysate; and at least one pharmaceutically acceptable carrier and/or excipient.

In some embodiments, the composition may comprise one or more components of a bacterial lysate from a bacterium having genomic DNA with a CpG abundance substantially similar to a CpG abundance in genomic DNA of a gut bacterium.

In various embodiments, the composition may comprise one or more synthetic analogues of one or more components of a bacterial lysate, wherein the bacterial lysate is from a bacterium having genomic DNA with a CpG abundance substantially similar to a CpG abundance in genomic DNA of a gut bacterium. In some embodiments, the composition is a pharmaceutical composition and the composition comprises one or more bacterial lysate from one or more species of bacteria having genomic DNA with a CpG abundance substantially similar to a CpG abundance in genomic DNA of a gut bacterium and a pharmaceutically acceptable carrier and/or excipient.

In some embodiments, the composition may comprise one or more components of a bacterial lysate from a bacterium comprising a Lipid A structure substantially similar to a Lipid A in B. thetaiotaomicron. In some embodiments, the composition is a pharmaceutical composition and the composition comprises one or more bacterial lysate from a species of bacteria that comprises a Lipid A structure substantially similar to a Lipid A in B. thetaiotaomicron and at least one pharmaceutically acceptable carrier and/or excipient.

In some embodiments, the composition may comprise one or more synthetic analogues of one or more components of a bacterial lysate from a bacterium having a Lipid A structure substantially similar to a Lipid A in B. thetaiotaomicron.

In some embodiments, the composition may comprise one or more components of a bacterial lysate from a bacterium comprising a lipoteichoic acid (LTA) having a structure substantially similar to the LTA found in F. prausnitzii. Lipoteichoic acid (LTA) is defined in the art as an alditolphosphate-containing polymer that is linked via a lipid anchor to the membrane in gram-positive bacteria. Five types of LTAs are broadly categorized into two groups: (Polyglycerolphosphate (Type I) and Complex LTAs (Type II, Type III, Type IV, and Type V). Accordingly, in some embodiments, the bacterium comprises a lipoteichoic acid (LTA) selected from any one of Type I, Type II, Type III, Type IV or Type V LTA, as described in Percy et al., (“Lipoteichoic acid synthesis and function in gram-positive bacteria” Annu. Rev. Microbiol. 2014, 68:81-100) the entire disclosure of which is incorporated herein by reference. In some embodiments, the composition may comprise one or more synthetic analogues of one or more components of a bacterial lysate from a bacterium having a lipoteichoic acid (LTA) structure substantially similar to a LTA in F. prausnitzii.

In some embodiments, the composition may comprise a bacterial lysate, one or more components of the bacterial lysate, or one or more synthetic analogues of one or more components of the bacterial lysate, wherein the bacterial lysate or components/synthetic analogues thereof contain one or more ligands that are capable of binding to toll-like receptor 2, toll-like receptor 4 and nucleotide-binding oligomerization domain 2 (NOD2) receptor on a target cell to a degree sufficient to activate a cellular response in the target cell.

In further embodiments the composition may comprise one or more of: (a) one or more bacterial lysates from one or more species of a Gram-positive bacterial cell and one or more bacterial lysates from one or more species of Gram-negative bacterial cell; (b) one or more components from one or more bacterial lysates from one or more species of a Gram-positive bacterial cell and one or more components from one or more bacterial lysates from one or more species of a Gram-negative bacterial cell; (c) one or more synthetic analogues of one or more components from one or more bacterial lysates from one or more species of a Gram-positive bacterial cell and one or more synthetic analogues of one or more components from one or more bacterial lysates from one or more species of a Gram-negative bacterial cell (d) one or more bacterial lysate from one or more species of bacteria having genomic DNA with a CpG abundance substantially similar to a CpG abundance in genomic DNA of a gut bacterium; (e) one or more synthetic analogues of one or more components of a bacterial lysate, wherein the bacterial lysate is from one or more species of bacteria having genomic DNA with a CpG abundance substantially similar to a CpG abundance in genomic DNA of a gut bacterium; (f) one or more bacterial lysate from a species of bacteria that comprises a Lipid A structure substantially similar to a Lipid A in B. thetaiotaomicron; or (g) one or more synthetic analogues of one or more components of a bacterial lysate from a species of bacteria that comprises a Lipid A structure substantially similar to a Lipid A in B. thetaiotaomicron. In some embodiments the composition is a pharmaceutical composition and further comprises at least at least one pharmaceutically acceptable carrier and/or excipient.

In various aspects, the composition comprises at least two of (a), (b), (c), (d), (e), (f) or (g) as described above. In various embodiments, the composition comprises at least one of (a) or (b) or (c); at least one of (d) or (e); and/or at least one of (f) or (g). In various aspects, the composition comprises at least one of (a) or (b) or (c); at least one of (d) or (e) and at least one of (f) or (g). In various aspects the composition comprises (a) and (d) and (f). In various aspects, the composition comprises (b) and (e) and (g). In various aspects, the composition comprises (c) and (e) and (g). In further aspects, the composition may comprise (a) and (e) and (g); (b) and (d) and (g); (c) and (d) and (g); (b) and (e) and (f); (c) and (e) and (f); (a) and (d) and (g); (b) and (d) and (f); (c) and (d) and (f); or (a) and (e) and (f).

(a) Lysates Prepared from One or More Species of Gram-Positive and Gram-Negative Bacteria

In various aspects, the composition comprises one or more bacterial lysates from one or more species of a Gram-positive bacterial cell and one or more bacterial lysates from one or more species of Gram-negative bacterial cell.

In some embodiments, the species of Gram-positive bacteria can comprise one or more of the species in Table A.

TABLE A

| | NCBI GenBank |
|---|---|
| Species | Accession Number |
| Actinomyces israelii | AF479270 |
| Actinomyces odontolyticus | ACYT010000123 |
| Bifidobacterium adolescentis | AAXD02000018 |
| Bifidobacterium aesculapii | NZ_BCFK00000000 |
| Bifidobacterium angulatum | ABYS02000004 |
| Bifidobacterium animalis | CP001606 |
| Bifidobacterium anseris | NZ_NMYC00000000 |
| Bifidobacterium aquikefiri | NR_148810 |
| Bifidobacterium asteroides | M58730 |
| Bifidobacterium biavatii | LC519989 |
| Bifidobacterium bifidum | ABQP01000027 |
| Bifidobacterium bohemicum | FJ858737 |
| Bifidobacterium bombi | NR_116178 |
| Bifidobacterium boum | MT742663 |
| Bifidobacterium breve | CP002743 |
| Bifidobacterium callitrichos | NZ_NWTX00000000 |
| Bifidobacterium catenulatum | ABXY01000019 |
| Bifidobacterium choerinum | NZ_CP018044 |
| Bifidobacterium commune | LK054489 |
| Bifidobacterium coryneforme | NZ_CP007287 |
| Bifidobacterium criceti | NZ_MVOH01000029 |
| Bifidobacterium crudilactis | AY952448 |
| Bifidobacterium cuniculi | NZ_JGYV01000038 |
| Bifidobacterium dentium | CP002743 |
| Bifidobacterium eulemuris | NR_148784 |
| Bifidobacterium gallicum | ABXB03000004 |
| Bifidobacterium gallinarum | MN918286 |
| Bifidobacterium hapali | KP718963 |
| Bifidobacterium imperatoris | NZ_CP071591 |
| Bifidobacterium indicum | M58737 |
| Bifidobacterium italicum | NZ_MVOG01000072 |
| Bifidobacterium kashiwanohense | AB491757 |
| Bifidobacterium lemurum | NZ_BDIS01000046 |
| Bifidobacterium longum | ABQQ01000041 |
| Bifidobacterium magnum | M58740 |
| Bifidobacterium margollesii | NZ_NMWU00000000 |
| Bifidobacterium merycicum | CADAXU000000000 |
| Bifidobacterium minimum | NZ_JGZD01000018 |
| Bifidobacterium mongoliense | NR_041686 |
| Bifidobacterium moukalabense | NZ_BJFB00000000 |
| Bifidobacterium myosotis | KP718942 |
| Bifidobacterium parmae | NZ_NMWT00000000 |
| Bifidobacterium pseudocatenulatum | ABXX02000002 |
| Bifidobacterium pseudolongum | NR_043442 |
| Bifidobacterium psychraerophilum | NZ_JGZI01000011 |
| Bifidobacterium pullorum | AY004278 |
| Bifidobacterium reuteri | NZ_RZUG00000000 |
| Bifidobacterium ruminantium | NZ_JAHPZN000000000 |
| Bifidobacterium saeculare | NR_029137 |
| Bifidobacterium saguini | CP071732 |
| Bifidobacterium scardovii | AJ307005 |
| Bifidobacterium subtile | CP062939 |
| Bifidobacterium thermacidophilum | NR_037049 |
| Bifidobacterium thermophilum | DQ340557 |
| Bifidobacterium tissieri | NR_147761 |
| Bifidobacterium tsurumiense | NR_041348 |
| Bifidobacterium vansinderenii | NZ_NEWD00000000 |
| Blautia coccoides | AB571656 |
| Blautia hansenii | ABYU02000037 |
| Blautia hominis | NR_163638 |
| Blautia hydrogenotrophica | ACBZ01000217 |
| Blautia marasmi | NZ_CABMKU000000000 |
| Blautia massiliensis | NZ_JACOGH010000024 |
| Blautia obeum | KY914474 |
| Blautia producta | AB600998 |
| Blautia schinkii | NR_026312 |
| Blautia sp. An249 | NZ_NFJL00000000 |
| Blautia sp. An46 | NZ_NFIF00000000 |
| Blautia sp. An81 | NZ_CAJFMH000000000 |
| Blautia sp. KLE 1732 | NZ_KE993452 |
| Blautia sp. Marseille-P2398 | LT161889 |
| Blautia sp. Marseille-P3087 | LT631515 |
| Blautia sp. Marseille-P3201T | LT623891 |
| Blautia sp. SF-50 | NZ_FMUW01000078 |
| Blautia sp. YL58 | KR364747 |
| Blautia wexlerae | EF036367 |
| Clostridium aceticum | NR_037126 |
| Clostridium acetireducens | NR_026179 |
| Clostridium acetobutylicum | NR_074511 |
| Clostridium acidisoli | NR_028898 |
| Clostridium aerotolerans | X76163 |
| Clostridium akagii | NR_025352 |
| Clostridium algidicarnis | NR_041746 |
| Clostridium amazonitimonense | LK021125 |
| Clostridium aminophilum | L04165 |
| Clostridium amygdalinum | AY353957 |
| Clostridium amylolyticum | NR_044386 |
| Clostridium arbusti | NR_116458 |
| Clostridium argentinense | NR_029232 |
| Clostridium asparagiforme | ACCJ01000522 |
| Clostridium aurantibutyricum | NR_044841 |
| Clostridium autoethanogenum | NR_119283 |
| Clostridium baratii | NR_029229 |
| Clostridium beijerinckii | NR_074434 |
| Clostridium bolteae | ABCC02000039 |
| Clostridium bornimense | NR_134005 |
| Clostridium botulinum | NC_010723 |
| Clostridium butyricum | ABDT01000017 |
| Clostridium cadaveris | AB542932 |
| Clostridium carboxidivorans | FR733710 |
| Clostridium cavendishii | NR_115711 |
| Clostridium celatum | X77844 |
| Clostridium celerecrescens | JQ246092 |
| Clostridium cellobioparum | MH708235 |
| Clostridium cellulolyticum | M87018 |
| Clostridium cellulosi | NR_044624 |
| Clostridium cellulovorans | NR_102875 |
| Clostridium chauvoei | NR_026013 |
| Clostridium chromiireducens | NR_122090 |
| Clostridium citroniae | ADLJ01000059 |
| Clostridium clariflavum | NR_041235 |
| Clostridium clostridioforme | NR_044715 |
| Clostridium cochlearium | NR_044717 |
| Clostridium cocleatum | NR_026495 |
| Clostridium colicanis | NR_028964 |
| Clostridium collagenovorans | NR_029246 |
| Clostridium coskatii | NZ_LROR01000105 |
| Clostridium cylindrosporum | NR_026492 |
| Clostridium dakarense | NZ_HG529480 |
| Clostridium diolis | NR_025542 |
| Clostridium disporicum | NR_026491 |
| Clostridium drakei | NR_044942 |
| Clostridium estertheticum | NR_042153 |
| Clostridium fallax | NR_044714 |
| Clostridium felsineum | AF270502 |
| Clostridium fimetarium | NR_024993 |
| Clostridium formicaceticum | NR_029267 |
| Clostridium frigidicarnis | NR_024919 |
| Clostridium gasigenes | NR_024945 |
| Clostridium glycyrrhizinilyticum | AB233029 |
| Clostridium grantii | NR_026131 |
| Clostridium haemolyticum | NR_024749 |
| Clostridium hiranonis | AB023970 |
| Clostridium homopropionicum | NR_026131 |
| Clostridium hungatei | AF020429 |
| Clostridium hydrogeniformans | NR_115712 |
| Clostridium hylemonae | AB023973 |
| Clostridium ihumii | NR_144705 |
| Clostridium indolis | AF028351 |
| Clostridium innocuum | M23732 |
| Clostridium intestinale | NR_029263 |
| Clostridium jeddahense | NR_144697 |
| Clostridium josui | AB011057 |
| Clostridium kluyveri | NR_074165 |
| Clostridium lavalense | EF564277 |
| Clostridium leptum | AJ305238 |
| Clostridium ljungdahlii | NR_074161 |
| Clostridium lundense | NR_043235 |
| Clostridium magnum | X77835 |
| Clostridium massiliodielmoense | NR_144739 |
| Clostridium mediterraneense | NZ_CABKWO010000001 |
| Clostridium merdae | NR_147400 |
| Clostridium methoxybenzovorans | BMAX01000001 |
| Clostridium methylpentosum | NR_029355 |
| Clostridium neonatale | MG030671 |
| Clostridium nigeriense | NZ_LT575471 |
| Clostridium novyi | NR_074343 |
| Clostridium oryzae | NR_134714 |
| Clostridium papyrosolvens | KC758692 |
| Clostridium paradoxum | NR_119334 |
| Clostridium paraputrificum | AB536771 |
| Clostridium pasteurianum | NR_104822 |
| Clostridium peptidivorans | NR_025019 |
| Clostridium perfringens | ABDW01000023 |
| Clostridium phoceensis | NZ_CABKUC010000004 |
| Clostridium polynesiense | NR_144690 |
| Clostridium polysaccharolyticum | NR_119085 |
| Clostridium populeti | NR_026103 |
| Clostridium puniceum | NR_026105 |
| Clostridium ragsdalei | DQ020022 |
| Clostridium roseum | NR_153749 |
| Clostridium saccharobutylicum | NR_036951 |
| Clostridium saccharogumia | DQ100445 |
| Clostridium saccharolyticum | CP002109 |
| Clostridium saccharoperbutylacetonicum | NR_036950 |
| Clostridium sartagoforme | NR_026490 |
| Clostridium saudiense | NR_144696 |
| Clostridium scatologenes | NR_118727 |
| Clostridium scindens | AF262238 |
| Clostridium senegalense | NR_125591 |
| Clostridium septicum | NR_026020 |
| Clostridium sphenoides | X73449 |
| Clostridium spiroforme | X73441 |
| Clostridium sporogenes | ABKW02000003 |
| Clostridium sporosphaeroides | NR_044835 |
| Clostridium stercorarium | NR_025100 |
| Clostridium straminisolvens | NR_024829 |
| Clostridium sulfidigenes | NR_044161 |
| Clostridium symbiosum | ADLQ01000114 |
| Clostridium tepidiprofundi | NR_044159 |
| Clostridium tepidum | NR_157639 |
| Clostridium termitidis | FR733680 |
| Clostridium tertium | Y18174 |
| Clostridium tetani | NC_004557 |
| Clostridium tetanomorphum | NR_043671 |
| Clostridium thermoalcaliphilum | NR_117153 |
| Clostridium thermobutyricum | NR_044718 |
| Clostridium thermosuccinogenes | Y18180 |
| Clostridium tunisiense | NR_115161 |
| Clostridium tyrobutyricum | NR_044718 |
| Clostridium uliginosum | NR_028920 |
| Clostridium ultunense | NZ_AZSU01000012 |
| Clostridium ventriculi | NR_026146 |
| Clostridium viride | NR_026204 |
| Collinsella aerofaciens | AAVN02000007 |
| Collinsella bouchesdurhonensis | NR_147379 |
| Collinsella ihuae | LN881598 |
| Collinsella intestinalis | ABXH02000037 |
| Collinsella phocaeensis | NR_147399 |
| Collinsella stercoris | ABXJ01000150 |
| Collinsella tanakaei | AB490807 |
| Collinsella vaginalis | NZ_LT838864 |
| Coprococcus comes | NR_044048 |
| Coprococcus eutactus | EF031543 |
| Coprococcus sp. HPP0048 | AGEW01000011 |
| Coprococcus sp. HPP0074 | NZ_KE150440 |
| Corynebacterium diphtheriae | NC_002935 |
| Corynebacterium doosanense | NR_116525 |
| Corynebacterium jeikeium | ACYW01000001 |
| Dorea formicigenerans | AAXA02000006 |
| Dorea longicatena | AJI32842 |
| Dorea sp. 5-2 | NZ_KE159793 |
| Dorea sp. AGR2135 | NZ_ATVU01000073 |
| Dorea sp. D27 | NZ_KQ236752 |
| Dorea sp. Marseille-P4003 | LT934499 |
| Eggerthella lenta | AF292375 |
| Eggerthella timonensis | NR_147380 |
| Enterococcus aquimarinus | NR_042375 |
| Enterococcus asini | NR_029337 |
| Enterococcus avium | AF133535 |
| Enterococcus caccae | AY943820 |
| Enterococcus canintestini | NR_042386 |
| Enterococcus canis | NR_044852 |
| Enterococcus casseliflavus | AEWT01000047 |
| Enterococcus cecorum | NR_024905 |
| Enterococcus columbae | NR_041708 |
| Enterococcus devriesei | NR_042389 |
| Enterococcus dispar | NR_024904 |
| Enterococcus durans | AJ276354 |
| Enterococcus faecalis | AE016830 |
| Enterococcus faecium | AMI57434 |
| Enterococcus gallinarum | AB269767 |
| Enterococcus gilvus | AY033814 |
| Enterococcus haemoperoxidus | NR_028795 |
| Enterococcus hermanniensis | NR_042897 |
| Enterococcus hirae | AF061011 |
| Enterococcus italicus | AEPV01000109 |
| Enterococcus malodoratus | NR_042055 |
| Enterococcus massiliensis | NR_144723 |
| Enterococcus moraviensis | NR_028794 |
| Enterococcus mundtii | NR_024906 |
| Enterococcus pallens | NR_043794 |
| Enterococcus pernyi | FJ555518 |
| Enterococcus phoeniculicola | NR_029040 |
| Enterococcus plantarum | NR_118050 |
| Enterococcus pseudoavium | NR_028705 |
| Enterococcus quebecensis | NR_117519 |
| Enterococcus raffinosus | FN600541 |
| Enterococcus ratti | NR_041933 |
| Enterococcus rivorum | NR_117043 |
| Enterococcus saccharolyticus | NR_041707 |
| Enterococcus silesiacus | NR_042405 |
| Enterococcus sulfureus | NR_041706 |
| Enterococcus termitis | NR_042406 |
| Enterococcus thailandicus | AY321376 |
| Enterococcus timonensis | LT576388 |
| Enterococcus ureasiticus | NR_117520 |
| Enterococcus ureilyticus | NR_125485 |
| Enterococcus villorum | NR_036921 |
| Enterococcus wangshanyuanii | NR_159231 |
| Eubacterium aggregans | NR_024926 |
| Eubacterium barkeri | NR_044661 |
| Eubacterium brachy | U13038 |
| Eubacterium callanderi | NR_024330 |
| Eubacterium cellulosolvens | AY178842 |
| Eubacterium combesii | MG334166 |
| Eubacterium coprostanoligenes | HM037995 |
| Eubacterium eligens | CP001104 |
| Eubacterium hallii | L34621 |
| Eubacterium infirmum | U13039 |
| Eubacterium limosum | CP002273 |
| Eubacterium nodatum | U13041 |
| Eubacterium oxidoreducens | NR_104737 |
| Eubacterium plexicaudatum | AF157058 |
| Eubacterium pyruvativorans | NR_042074 |
| Eubacterium ramulus | AJ011522 |
| Eubacterium rectale | FP929042 |
| Eubacterium ruminantium | NR_024661 |
| Eubacterium saphenum | NR_026031 |
| Eubacterium siraeum | ABCA03000054 |
| Eubacterium sulci | GU413139 |
| Eubacterium uniforme | NR_104842 |
| Eubacterium ventriosum | L34421 |
| Eubacterium xylanophilum | L34628 |
| Eubacterium yurii | AEES01000073 |
| Faecalibacterium prausnitzii | ACOP02000011 |
| Faecalibacterium sp. An121 | NZ_NFLM01000051 |
| Faecalibacterium sp. An122 | NZ_NFLL01000070 |
| Faecalibacterium sp. An192 | NZ_NFKB01000132 |
| Faecalibacterium sp. An58 | NZ_NFHX01000066 |
| Faecalibacterium sp. An77 | NFHK01000001 |
| Faecalibaculum rodentium | NR_146011 |
| Holdemania filiformis | Y11466 |
| Holdemania massiliensis | NR_125628 |
| Holdemania sp. Marseille-P2844 | LT576390 |
| Lactobacillus acetotolerans | NR_044699 |
| Lactobacillus acidifarinae | NR_042242 |
| Lactobacillus acidipiscis | NR_024718 |
| Lactobacillus acidophilus | CP000033 |
| Lactobacillus agilis | NR_044700 |
| Lactobacillus algidus | NR_028617 |
| Lactobacillus alimentarius | NR_044701 |
| Lactobacillus allii | NR_159082 |
| Lactobacillus amylolyticus | ADNY01000006 |
| Lactobacillus amylophilus | NR_044702 |
| Lactobacillus amylotrophicus | NR_042511 |
| Lactobacillus amylovorus | CP002338 |
| Lactobacillus animalis | NR_041610 |
| Lactobacillus antri | ACLL01000037 |
| Lactobacillus apinorum | NR_126247 |
| Lactobacillus apis | NR_125702 |
| Lactobacillus apodemi | NR_042367 |
| Lactobacillus aquaticus | NR_115847 |
| Lactobacillus aviarius | NR_044703 |
| Lactobacillus backii | NR_114385 |
| Lactobacillus bifermentans | NR_104926 |
| Lactobacillus bombicola | NR_136436 |
| Lactobacillus brantae | NR_125575 |
| Lactobacillus brevis | EU194349 |
| Lactobacillus buchneri | ACGH01000101 |
| Lactobacillus cacaonum | NR_042677 |
| Lactobacillus camelliae | NR_041456 |
| Lactobacillus capillatus | NR_041655 |
| Lactobacillus casei | CP000423 |
| Lactobacillus ceti | NR_042539 |
| Lactobacillus coleohominis | ACOH01000030 |
| Lactobacillus collinoides | AB005893 |
| Lactobacillus composti | NR_041509 |
| Lactobacillus concavus | NR_043105 |
| Lactobacillus coryniformis | NR_044705 |
| Lactobacillus crispatus | ACOG01000151 |
| Lactobacillus crustorum | NR_042533 |
| Lactobacillus curieae | NR_109538 |
| Lactobacillus curvatus | NR_042437 |
| Lactobacillus delbrueckii | NR_029106 |
| Lactobacillus dextrinicus | AB627845 |
| Lactobacillus diolivorans | AB429369 |
| Lactobacillus equi | NR_028623 |
| Lactobacillus equicursoris | NR_112652 |
| Lactobacillus equigenerosi | NR_041566 |
| Lactobacillus fabifermentans | NR_042676 |
| Lactobacillus farciminis | NR_044707 |
| Lactobacillus farraginis | NR_041467 |
| Lactobacillus fermentum | CP002033 |
| Lactobacillus floricola | NR_113001 |
| Lactobacillus florum | NR_112911 |
| Lactobacillus fructivorans | NR_036789 |
| Lactobacillus frumenti | HM755669 |
| Lactobacillus fuchuensis | AB063479 |
| Lactobacillus futsali | NR_165758 |
| Lactobacillus gallinarum | AB008208 |
| Lactobacillus gasseri | AB008209 |
| Lactobacillus gastricus | AICN01000060 |
| Lactobacillus ghanensis | NR_043896 |
| Lactobacillus gigeriorum | AYZO01000010 |
| Lactobacillus ginsenosidimutans | NR_132607 |
| Lactobacillus gorillae | NR_134066 |
| Lactobacillus graminis | AB289145 |
| Lactobacillus hammesii | AB512777 |
| Lactobacillus hamsteri | AB289146 |
| Lactobacillus harbinensis | AB194126 |
| Lactobacillus hayakitensis | AB267406 |
| Lactobacillus heilongjiangensis | NR_109370 |
| Lactobacillus helsingborgensis | EF187242 |
| Lactobacillus helveticus | ACLM01000202 |
| Lactobacillus herbarum | KR706503 |
| Lactobacillus hilgardii | ACGP01000200 |
| Lactobacillus hokkaidonensis | AB721548 |
| Lactobacillus hominis | FR681902 |
| Lactobacillus homohiochii | AF429598 |
| Lactobacillus hordei | NR_044394 |
| Lactobacillus iners | AEKJ01000002 |
| Lactobacillus ingluviei | AB289164 |
| Lactobacillus intestinalis | AB260945 |
| Lactobacillus jensenii | ACQD01000066 |
| Lactobacillus johnsonii | AE017198 |
| Lactobacillus kalixensis | NR_029083 |
| Lactobacillus kefiranofaciens | NR_042440 |
| Lactobacillus kefiri | NR_042230 |
| Lactobacillus kimbladii | JX099548 |
| Lactobacillus kimchicus | LC480811 |
| Lactobacillus kimchiensis | HQ90650 |
| Lactobacillus kisonensis | AB366388 |
| Lactobacillus kitasatonis | AB107637 |
| Lactobacillus koreensis | FJ904277 |
| Lactobacillus kullabergensis | EF187241 |
| Lactobacillus kunkeel | AB498039 |
| Lactobacillus lindneri | AB512778 |
| Lactobacillus malefermentans | AB680994 |
| Lactobacillus mali | AB690200 |
| Lactobacillus manihotivorans | AF000162 |
| Lactobacillus mellifer | EF187245 |
| Lactobacillus mellis | EF187244 |
| Lactobacillus melliventris | EF187243 |
| Lactobacillus mindensis | AB626076 |
| Lactobacillus mixtipabuli | AB894863 |
| Lactobacillus mucosae | FR693800 |
| Lactobacillus murinus | NR_042231 |
| Lactobacillus nagelii | AB162131 |
| Lactobacillus namurensis | AB626072 |
| Lactobacillus nantensis | AB626067 |
| Lactobacillus nasuensis | NR_113303 |
| Lactobacillus nodensis | NR_041629 |
| Lactobacillus odoratitofui | AB365975 |
| Lactobacillus oeni | NR_043095 |
| Lactobacillus oligofermentans | NR_043148 |
| Lactobacillus oris | AEKL01000077 |
| Lactobacillus oryzae | NR_114339 |
| Lactobacillus otakiensis | BASH01000001 |
| Lactobacillus ozensis | NR_113194 |
| Lactobacillus panis | NR_026310 |
| Lactobacillus pantheris | MK314721 |
| Lactobacillus parabrevis | NR_042456 |
| Lactobacillus parabuchneri | NR_041294 |
| Lactobacillus paracasei | ABQV01000067 |
| Lactobacillus paracollinoides | NR_112756 |
| Lactobacillus parafarraginis | JH414901 |
| Lactobacillus parakefiri | NR_029039 |
| Lactobacillus paralimentarius | NR_036879 |
| Lactobacillus paraplantarum | MT622658 |
| Lactobacillus pasteurii | NZ_PUFQ01000022 |
| Lactobacillus paucivorans | NR_116943 |
| Lactobacillus pentosiphilus | NR_158060 |
| Lactobacillus pentosus | JN813103 |
| Lactobacillus perolens | NR_029360 |
| Lactobacillus plantarum | ACGZ02000033 |
| Lactobacillus pobuzihii | BJYB01000001 |
| Lactobacillus pontis | HM218420 |
| Lactobacillus psittaci | NR_125811 |
| Lactobacillus rapi | BKAM01000001 |
| Lactobacillus rennini | FR714836 |
| Lactobacillus reuteri | NG_048296 |
| Lactobacillus rhamnosus | ABWJ01000068 |
| Lactobacillus rogosae | GU269544 |
| Lactobacillus rossiae | KE386820 |
| Lactobacillus ruminis | ACGS02000043 |
| Lactobacillus saerimneri | KE383994 |
| Lactobacillus sakei | DQ989236 |
| Lactobacillus salivarius | AEBA01000145 |
| Lactobacillus sanfranciscensis | KY306448 |
| Lactobacillus saniviri | AB602569 |
| Lactobacillus satsumensis | NR_028658 |
| Lactobacillus secaliphilus | NR_042523 |
| Lactobacillus selangorensis | JQAT01000001 |
| Lactobacillus senioris | AB602570 |
| Lactobacillus senmaizukei | AYZH01000001 |
| Lactobacillus sharpeae | BBAC01000005 |
| Lactobacillus shenzhenensis | NR_118471 |
| Lactobacillus silagei | NR_114388 |
| Lactobacillus silagincola | NR_158059 |
| Lactobacillus siliginis | BJUD01000001 |
| Lactobacillus similis | NR_112645 |
| Lactobacillus spicheri | AJ534844 |
| Lactobacillus sucicola | NR_112785 |
| Lactobacillus suebicus | GU174474 |
| Lactobacillus sunkii | NR_041656 |
| Lactobacillus taiwanensis | EU647675 |
| Lactobacillus thailandensis | NR_041456 |
| Lactobacillus timonensis | NZ_LT964776 |
| Lactobacillus tucceti | NR_042194 |
| Lactobacillus ultunensis | ACGU01000081 |
| Lactobacillus uvarum | NR_115308 |
| Lactobacillus vaccinostercus | NR_112541 |
| Lactobacillus vaginalis | ACGV01000168 |
| Lactobacillus versmoldensis | NR_028990 |
| Lactobacillus vini | NR_042196 |
| Lactobacillus wasatchensis | NR_147709 |
| Lactobacillus xiangfangensis | MH605355 |
| Lactobacillus zeae | NR_037122 |
| Lactobacillus zymae | MN188052 |
| Roseburia faecis | AY305310 |
| Roseburia hominis | AJ270482 |
| Roseburia intestinalis | FP929050 |
| Roseburia inulinivorans | AJ270473 |
| Roseburia sp. 499 | MJID01000003 |
| Rothia aeria | DQ673320 |
| Rothia dentocariosa | ADDW01000024 |
| Rothia mucilaginosa | ACVO01000020 |
| Rothia nasimurium | NR_025310 |
| Ruminococcus albus | AY445600 |
| Ruminococcus bicirculans | WQNR01000149 |
| Ruminococcus bromii | EU266549 |
| Ruminococcus callidus | NR_029160 |
| Ruminococcus champanellensis | FP929052 |
| Ruminococcus faecis | NR_116747 |
| Ruminococcus flavefaciens | NR_025931 |
| Ruminococcus gauvreauii | NR_044265 |
| Ruminococcus gnavus | X94967 |
| Ruminococcus lactaris | ABOU02000049 |
| Ruminococcus sp. 5_1_39BFAA | ACII01000172 |
| Ruminococcus sp. AT10 | LN912997 |
| Ruminococcus sp. DSM 100440 | KT156811 |
| Ruminococcus sp. FC2018 | KK211351 |
| Ruminococcus sp. HUN007 | JOOA00000000 |
| Ruminococcus sp. NK3A76 | GU324399 |
| Ruminococcus sp. YE71 | AY367006 |
| Ruminococcus sp. YE78 | KF156793 |
| Ruminococcus torques | AAVP02000002 |
| Streptococcus acidominimus | NR_104972 |
| Streptococcus agalactiae | AAJO01000130 |
| Streptococcus anginosus | AECT01000011 |
| Streptococcus australis | AEQR01000024 |
| Streptococcus azizii | KM609118 |
| Streptococcus bovimastitidis | LZDD01000002 |
| Streptococcus caballi | EF364097 |
| Streptococcus canis | AJ413203 |
| Streptococcus castoreus | AJ606047 |
| Streptococcus constellatus | AY277942 |
| Streptococcus criceti | AB026123 |
| Streptococcus cristatus | AEVC01000028 |
| Streptococcus cuniculi | HG793791 |
| Streptococcus devriesei | AJ564067 |
| Streptococcus didelphis | AF176100 |
| Streptococcus dysgalactiae | AP010935 |
| Streptococcus entericus | AM269537 |
| Streptococcus equi | AB002515 |
| Streptococcus equinus | AEVB01000043 |
| Streptococcus ferus | AB105868 |
| Streptococcus gallolyticus | FR824043 |
| Streptococcus gordonii | NC_009785 |
| Streptococcus halotolerans | KU864999 |
| Streptococcus henryi | AQYA01000001 |
| Streptococcus himalayensis | BMJN01000003 |
| Streptococcus hyovaginalis | AB238622 |
| Streptococcus ictaluri | DQ462421 |
| Streptococcus infantarius | ABJK02000017 |
| Streptococcus infantis | AFNN01000024 |
| Streptococcus iniae | AB470235 |
| Streptococcus intermedius | NR_028736 |
| Streptococcus lutetiensis | NR_037096 |
| Streptococcus macacae | AB195308 |
| Streptococcus macedonicus | AB563236 |
| Streptococcus marimammalium | AJ634751 |
| Streptococcus marmotae | CP015196 |
| Streptococcus massiliensis | AY769997 |
| Streptococcus merionis | AM396401 |
| Streptococcus minor | AM269579 |
| Streptococcus mitis | AM157420 |
| Streptococcus mutans | AAP010655 |
| Streptococcus oralis | ADMV01000001 |
| Streptococcus orisasini | AB668377 |
| Streptococcus orisratti | AB238813 |
| Streptococcus ovis | AM269567 |
| Streptococcus pantholopis | KU877326 |
| Streptococcus parasanguinis | AEKM01000012 |
| Streptococcus parauberis | AB175054 |
| Streptococcus pasteurianus | AP012054 |
| Streptococcus peroris | AEVF01000016 |
| Streptococcus phocae | AF235052 |
| Streptococcus pluranimalium | AB701574 |
| Streptococcus plurextorum | AM774228 |
| Streptococcus pneumoniae | AE008537 |
| Streptococcus porci | AM941160 |
| Streptococcus porcinus | EF121439 |
| Streptococcus pseudopneumoniae | FJ827123 |
| Streptococcus pseudoporcinus | AENS01000003 |
| Streptococcus pyogenes | AE006496 |
| Streptococcus ratti | X58304 |
| Streptococcus salivarius | AGBV01000001 |
| Streptococcus sanguinis | NR_074974 |
| Streptococcus sinensis | AF432857 |
| Streptococcus sobrinus | AB294731 |
| Streptococcus suis | FM252032 |
| Streptococcus thermophilus | CP000419 |
| Streptococcus thoraltensis | FN377815 |
| Streptococcus timonensis | MN577302 |
| Streptococcus uberis | HQ391900 |
| Streptococcus urinalis | DQ303194 |
| Streptococcus varani | CTEN00000001 |
| Streptococcus vestibularis | AY188353 |

The species of Gram-positive bacteria can, in some embodiments, comprise Faecalibacterium prausnitzii (F. prausnitzii), Lactobacillus johnsonii (L. johnsonii), Enterococcus faecalis (E. faecalis), Enterococcus faecium (E. faecium), Enterococcus gallinarum (E. gallinarum), Enterococcus hirae (E. hirae), Blautia producta (B. producta), Clostridium bolteae (C. bolteae), Bifidobacterium pseudolongum (B. pseudolongum), Lactobacillus acidophilus (L. acidophilus), or any combination thereof. In some embodiments, the species of Gram-positive bacteria comprises F. prausnitzii, B. producta, or any combination thereof. In some embodiments, the species of Gram-positive bacteria comprises F. prausnitzii. In some embodiments, the species of Gram-positive bacteria comprises L. johnsonii. In some embodiments, the species of Gram-positive bacteria comprises E. faecalis. In some embodiments, the species of Gram-positive bacteria comprises E. faecium. In some embodiments, the species of Gram-positive bacteria comprises E. gallinarum. In some embodiments, the species of Gram-positive bacteria comprises E. hirae. In some embodiments, the species of Gram-positive bacteria comprises B. producta. In some embodiments, the species of Gram-positive bacteria comprises C. bolteae. In some embodiments, the species of Gram-positive bacteria comprises B. pseudolongum. In some embodiments, the species of Gram-positive bacteria comprises L. acidophilus.

In various embodiments, the one or more species of Gram-positive bacterial cells comprise lipoteichoic acid (LTA), a major constituent of the cell wall of gram-positive bacteria, having a structure substantially similar to the LTA found in F. prausnitzii. In various aspects, the lipoteichoic acid (LTA) comprises an alditolphosphate-containing polymer that is linked via a lipid anchor to the membrane in gram-positive bacteria. As noted above, the LTA may comprise any one of Type I, Type II, Type III, Type IV, or Type V LTA as described in Percy et al., (“Lipoteichoic acid synthesis and function in gram-positive bacteria.” Annu. Rev. Microbiol. 2014, 68:81-100) which is incorporated herein by reference in its entirety. In additional embodiments, the one or more Gram-positive bacterial lysates may contain ligands that are capable of binding to toll-like receptor 2, toll-like receptor 4, or nucleotide-binding oligomerization domain 2 (NOD2) on a target cell, such binding being sufficient to activate a cellular response in the target cell

In some embodiments, the species of Gram-negative bacteria can comprise one or more of the bacteria in Table B.

TABLE B

| | NCBI GenBank |
|---|---|
| Species | Accession Number |
| Akkermansia sp. KLE1605 | NZ_KV441801 |
| Akkermansia sp. KLE1797 | NZ_KQ968641 |
| Akkermansia sp. KLE1798 | NZ_KQ971100 |
| Alistipes finegoldii | NR_043064 |
| Alistipes ihumii | CAJJIG000000000 |
| Alistipes indistinctus | AB490804 |
| Alistipes inops | NR_145882 |
| Alistipes obesi | KX198133 |
| Alistipes onderdonkii | NR_043318 |
| Alistipes putredinis | ABFK02000017 |
| Alistipes senegalensis | NZ_UYXI00000000 |
| Alistipes shahii | FP929032 |
| Alistipes sp. AL-1 | NZ_LN609288 |
| Alistipes sp. An116 | NZ_NFLR00000000 |
| Alistipes sp. An31A | NZ_NFIO00000000 |
| Alistipes sp. An54 | NZ_NFIA00000000 |
| Alistipes sp. An66 | NZ_NFHT00000000 |
| Alistipes sp. CHKCI003 | NZ_FCNT00000000 |
| Alistipes sp. cv1 | NZ_OEPW00000000 |
| Alistipes sp. HGB5 | AENZ01000082 |
| Alistipes sp. Marseille-P2431 | NZ_LT559262 |
| Alistipes sp. ZOR0009 | NZ_JTLD01000125 |
| Alistipes timonensis | NZ_CAJTAZ000000000 |
| Bacteroides acidifaciens | NR_028607 |
| Bacteroides barnesiae | NR_041446 |
| Bacteroides bouchesdurhonensis | NZ_LT707025 |
| Bacteroides caccae | EU136686 |
| Bacteroides caecimuris | NZ_VIRD00000000 |
| Bacteroides cellulosilyticus | ACCH01000108 |
| Bacteroides clarus | AFBM01000011 |
| Bacteroides congonensis | MZ701981 |
| Bacteroides coprocola | ABIY02000050 |
| Bacteroides coprophilus | ACBW01000012 |
| Bacteroides coprosuis | NR_112934 |
| Bacteroides cutis | NZ_OEST00000000 |
| Bacteroides dorei | ABWZ01000093 |
| Bacteroides eggerthii | ACWG01000065 |
| Bacteroides faecichinchillae | NZ_FQVD01000088 |
| Bacteroides faecis | GQ496624 |
| Bacteroides finegoldii | AB222699 |
| Bacteroides fluxus | AFBN01000029 |
| Bacteroides fragilis | AP006841 |
| Bacteroides gallinarum | NR_041448 |
| Bacteroides graminisolvens | AB363973 |
| Bacteroides helcogenes | CP002352 |
| Bacteroides ihuae | NZ_FNVX00000000 |
| Bacteroides intestinalis | ABJL02000006 |
| Bacteroides luti | NR_125463 |
| Bacteroides massiliensis | AB200226 |
| Bacteroides mediterraneensis | NZ_LT635798 |
| Bacteroides neonati | NZ_HG726036 |
| Bacteroides nordii | NR_043017 |
| Bacteroides oleiciplenus | AB547644 |
| Bacteroides ovatus | ACWH01000036 |
| Bacteroides paurosaccharolyticus | NR_113071 |
| Bacteroides plebeius | AB200218 |
| Bacteroides propionicifaciens | AB264625 |
| Bacteroides pyogenes | NR_041280 |
| Bacteroides reticulotermitis | NZ_JACIER000000000 |
| Bacteroides salanitronis | CP002530 |
| Bacteroides salyersiae | EU136690 |
| Bacteroides sartorii | KE159493 |
| Bacteroides stercorirosoris | AB574479 |
| Bacteroides stercoris | ABFZ02000022 |
| Bacteroides thetaiotaomicron | NR_074277 |
| Bacteroides timonensis | NZ_CBVI000000000 |
| Bacteroides uniformis | AB050110 |
| Bacteroides vulgatus | CP000139 |
| Bacteroides xylanisolvens | ADKP01000087 |
| Bacteroides xylanolyticus | DQ497992 |
| Bilophila sp. 4_1_30 | JH114231 |
| Bilophila wadsworthia | ADCP01000166 |
| Haemophilus aegyptius | AFBC01000053 |
| Haemophilus haemoglobinophilus | NR_042877 |
| Haemophilus haemolyticus | JN175335 |
| Haemophilus influenzae | AADP01000001 |
| Haemophilus massiliensis | NR_149208 |
| Haemophilus paracuniculus | NR_044751 |
| Haemophilus parahaemolyticus | GU561425 |
| Haemophilus parainfluenzae | AEWU01000024 |
| Haemophilus paraphrohaemolyticus | M75076 |
| Haemophilus pittmaniae | NR_025423 |
| Haemophilus quentini | AF224307 |
| Haemophilus sputorum | AFNK01000005 |
| Odoribacter laneus | AB490805 |
| Odoribacter splanchnicus | CP002544 |
| Parabacteroides chartae | NR_109439 |
| Parabacteroides chinchillae | NR_113208 |
| Parabacteroides distasonis | CP042285 |
| Parabacteroides goldsteinii | AY974070 |
| Parabacteroides gordonii | AB470344 |
| Parabacteroides johnsonii | ABYH01000014 |
| Parabacteroides merdae | EU136685 |
| Parabacteroides sp. 2_1_7 | KQ236092 |
| Parabacteroides sp. 20_3 | QSQL01000049 |
| Parabacteroides sp. An277 | NFJB01000083 |
| Parabacteroides sp. ASF519 | KB822574 |
| Parabacteroides sp. AT13 | NQMH01000003 |
| Parabacteroides sp. CT06 | CP022754 |
| Parabacteroides sp. D13 | GG698759 |
| Parabacteroides sp. D25 | JH976523 |
| Parabacteroides sp. D26 | KQ236102 |
| Parabacteroides sp. HGS0025 | KQ033911 |
| Parabacteroides sp. Marseille-P3160 | LT799418 |
| Parabacteroides sp. Marseille-P3668 | LT960514 |
| Parabacteroides sp. Marseille-P3763 | LT896587 |
| Parabacteroides sp. SN4 | LN899828 |
| Parabacteroides timonensis | NZ_LT669941 |
| Prevotella aff. ruminicola Tc2-24 | FOIQ01000008 |
| Prevotella albensis | NR_025300 |
| Prevotella amnii | AB547670 |
| Prevotella aurantiaca | NR_112878 |
| Prevotella baroniae | NR_043224 |
| Prevotella bergensis | ACKS01000100 |
| Prevotella bivia | ADFO01000096 |
| Prevotella brevis | NR_041954 |
| Prevotella bryantii | EU330891 |
| Prevotella buccae | ACRB01000001 |
| Prevotella buccalis | JN867261 |
| Prevotella conceptionensis | CAJPPB010000001 |
| Prevotella copri | ACBX02000014 |
| Prevotella corporis | L16465 |
| Prevotella dentalis | AB547678 |
| Prevotella dentasini | NR_112848 |
| Prevotella denticola | CP002589 |
| Prevotella disiens | AEDO01000026 |
| Prevotella enoeca | NR_025281 |
| Prevotella falsenii | NR_041684 |
| Prevotella fusca | MT795738 |
| Prevotella histicola | JN867315 |
| Prevotella ihumii | LT707005 |
| Prevotella intermedia | AF414829 |
| Prevotella jejuni | NR_109628 |
| Prevotella lascolaii | LT556060 |
| Prevotella loescheii | JN867231 |
| Prevotella maculosa | AGEK01000035 |
| Prevotella marshii | AEEI01000070 |
| Prevotella melaninogenica | CP002122 |
| Prevotella micans | AGWK01000061 |
| Prevotella multiformis | AEWX01000054 |
| Prevotella multisaccharivorax | AFJE01000016 |
| Prevotella nanceiensis | JN867228 |
| Prevotella nigrescens | AFPX01000069 |
| Prevotella oralis | AEPE01000021 |
| Prevotella oris | ADDV01000091 |
| Prevotella oryzae | LC005303 |
| Prevotella oulorum | L16472 |
| Prevotella pallens | AFPY01000135 |
| Prevotella paludivivens | NR_040924 |
| Prevotella phocaeensis | LT160626 |
| Prevotella pleuritidis | NR_041541 |
| Prevotella ruminicola | CP002006 |
| Prevotella saccharolytica | NR_113320 |
| Prevotella salivae | AB108826 |
| Prevotella scopos | NR_114305 |
| Prevotella shahii | NR_024815 |
| Prevotella stercorea | AB244774 |
| Prevotella timonensis | ADEF01000012 |
| Prevotella veroralis | ACVA01000027 |
| Veillonella atypica | AEDS01000059 |
| Veillonella denticariosi | EF185167 |
| Veillonella dispar | ACIK02000021 |
| Veillonella infantium | LC191258 |
| Veillonella magna | EU096495 |
| Veillonella montpellierensis | AF473836 |
| Veillonella parvula | ADFU01000009 |
| Veillonella rodentium | AY514996 |
| Veillonella rogosae | EF108443 |
| Veillonella seminalis | AY211542 |
| Veillonella tobetsuensis | AB679109 |

The species of Gram-negative bacteria can, in some embodiments, comprise Bacteroides thetaiotaomicron (B. thetaiotaomicron), Bacteroides vulgatus (B. vulgatus), Bacteroides ovatus (B. ovatus), Bacteroides uniformus (B. uniformus), Prevotella. copri (P. copri), Akkermansia muciniphila (A. muciniphila), or any combination thereof. In various embodiments, the species of Gram-negative bacteria comprises B. thetaiotaomicron, B. vulgatus, or any combination thereof. In some embodiments, the species of Gram-negative bacteria comprises B. thetaiotaomicron. In some embodiments, the Gram-negative bacteria comprise B. vulgatus. In some embodiments, the Gram-negative bacteria comprise B. ovatus. In some embodiments, the Gram-negative bacteria comprise B. uniformus. In some embodiments, the species of Gram-negative bacteria comprises P. copri. In some embodiments, the species of Gram-negative bacteria comprises A. muciniphila.

In additional embodiments, the one or more Gram-negative bacterial lysates may contain ligands that are capable of binding to toll-like receptor 2 (TLR2), toll-like receptor 4 (TLR4), or nucleotide-binding oligomerization domain 2 (NOD2) on a target cell, such binding being sufficient to activate a cellular response in the target cell

(b) A Component of Bacterial Lysate from One or More Species of Gram-Positive and/or Gram-Negative Bacterium.

In some embodiments, the composition can comprise one or more components obtained from the lysate of one or more species of a Gram-positive bacteria cell and/or one or more species of Gram-negative bacteria cell. The one or more species of Gram-positive and/or Gram-negative bacteria can be selected from those described above.

(c) A Synthetic Analogue of a Component of Bacterial Lysate from One or More Species Gram-Positive and/or a Gram-Negative Bacterium.

In some embodiments, the composition can comprise one or more synthetic analogues of one or more components obtained from a bacterial lysate from one or more species of Gram-positive and/or Gram-negative bacteria. In some embodiments, the one or more synthetic analogues can be in addition to the one or more lysates of the Gram-positive and/or Gram-negative bacteria. In some embodiments, the one or more synthetic analogues can replace the one or more lysates of the Gram-positive and/or Gram-negative bacteria.

(d) or (e) Lysates Prepared from One or More Species of Bacteria Having Nucleic Acids which Induce Host Innate Immune Pathways or a Synthetic Analogue of a Component Thereof.

An essential feature of mammalian innate immune cells is the ability to sense microbial nucleic acids. DNA appears to be a critical for gut microbiota lysate-induced innate immune cell activation. Three major receptors have been described in mammalian cells which can detect microbial DNA: 1) Toll-like receptor 9 (TLR9); 2) absent in melanoma 2 (AIM2); and cyclic-GMP-AMP synthase (cGAS). With regard to the former, TLR9 recognizes and is activated by unmethylated cytosine-phosphate-guanine (CpG) dinucleotides, which are relatively common in bacterial genomes. As such, CpG abundance provides an easily quantifiable surrogate for immunogenicity of a given bacteria's genomic DNA.

In various embodiments, the composition comprises one or more bacterial lysates from one or more species of bacteria having genomic DNA having a CpG abundance substantially similar to CpG abundance in the genomic DNA of a gut bacterium; or one or more synthetic analogues of one or more components of a lysate prepared from one or more species of bacteria having genomic DNA having a CpG abundance substantially similar to CpG abundance in the genomic DNA of a gut bacterium.

The phrase “CpG abundance,” as used herein, refers to the frequency of occurrences where a cytosine nucleotide is adjacent to a guanine nucleotide in the linear sequence of bases in the 5′ to 3′ direction in the genomic DNA of a bacterium. In various embodiments, lysate is prepared from bacteria having less than 1 million, less than 750,000 or less than 500,000 CpG motifs or occurrences per genome. In various embodiments, the composition comprises lysate from bacteria having from about 100,000 to 1,000,000 CpGs per genome, from about 100,000 to about 750,000 CpGs per genome, from about 100,000 to about 500,000 CpGs per genome or from about 200,000 to about 500,000 CpGs per genome.

In various embodiments, lysate is prepared from one or more species of bacteria having a CpG abundance substantially similar to a CpG abundance in genomic DNA of a gut bacterium. The species of gut bacteria can comprise F. prausnitzii, B. thetaiotaomicron, B. producta, B. vulgatus, B. ovatus, B. uniformus, P. copri, A. muciniphila, L. johnsonii, E. faecium, E. faecalis, E. gaffinarum, E. hirae, C. bolteae, B. pseudolongum, L. acidophilus, or any combination thereof. In various embodiments, the one or more species of bacteria having genomic DNA with a CpG abundance substantially similar to a CpG abundance in genomic DNA of a gut bacterium is F. prausnitzi, B. thetaiotaomicron, B. producta, B. vulgatus, or any combination thereof. For example, in some embodiments, the species of bacteria comprises F. prausnitzii, B. thetaiotaomicron, or any combination thereof. In some embodiments, the species of bacteria comprises F. prausnitzii. In some embodiments, the species of bacteria comprises B. thetaiotaomicron. In some embodiments, the species of bacteria comprises B. vulgatus. In some embodiments, the species of bacteria comprise B. ovatus. In some embodiments, the species of bacteria comprises B. uniformus. In some embodiments, the species of bacteria comprises P. copri. In some embodiments, the species of bacteria comprises A. muciniphila. In some embodiments, the species of bacteria comprises L. johnsonii. In some embodiments, the species of bacteria comprises E. faecium. In some embodiments, the species of bacteria comprises E. faecalis. In some embodiments, the species of bacteria comprises E. gaffinarum. In some embodiments, the species of bacteria comprises E. hirae. In some embodiments, the species of bacteria comprises C. bolteae. In some embodiments, the species of bacteria comprises B. pseudolongum. In some embodiments, the species of bacteria comprises L. acidophilus.

In various embodiments, lysate is prepared from one or more species of bacteria having a CpG abundance less than that found in bacteria in Coley's toxin (e.g., S. marcescens or S. pyogenes). For example, in various embodiments, lysate is prepared from bacteria having a CpG abundance 10% less than, 20% less than, 30% less than, 40% less than, 50% less than, 60% less than, 70% less than, 80% less than, or 90% less than the CpG abundance in Coley's toxin.

When used herein, “CpG abundance substantially similar” means within 50% greater than or less than a measured value of CpG abundance in the gut bacteria (such as F. prausnitzii or B. thetaiotaomicron). CpG abundance can be determined by methods known to those of skill in the art. For example, various bioinformatic tools such as genomic island related databases can be used to scan a complete genome and identify the frequency of CpG.

(f) or (g) Lysates Prepared from One or More Species of Bacteria Comprising a Lipid A Structure Substantially Similar to a Lipid A in B. thetaiotaomicron or a Synthetic Analogue of a Component Thereof.

In various aspects, the composition comprises one or more lysates from one or more species of bacteria having a Lipid A with a structure substantially similar to a Lipid A in B. thetaiotaomicron. Non-limiting exemplary species of bacteria that may have a structurally similar Lipid A to the Lipid A in B. thetaiotaomicron include Akkermansia spp, Parabacteroides spp, and Prevotella spp, As used herein, the term “substantially similar” refers to a compound having the same structure or nearly the same structure as the Lipid A in B. thetaiotaomicron. In various embodiments, a “substantially similar” structure can be determined using standard methods in the art such as mass spectrometry (e.g., MALDI-TOF mass spectrometry). The structure of the Lipid A of the bacteria may comprise a mono-phosphoryl lipid A. In various embodiments, the lipid A of the bacteria used to prepare the lysate can comprise 5-6 acyl chains. In some embodiments, the composition can comprise a lysate of a bacteria having a mono-phosphoryl lipid A with 5-6 acyl chains.

In various embodiments, the composition comprises a synthetic analogue of one or more components obtained from the lysate of the one or more species of bacteria having a similar Lipid A structure to B. thetaiotaomicron. For example, in various embodiments, the composition can comprise a natural or synthetic mono-phosphoryl lipid A with 5-6 acyl chains.

In various embodiments, the composition comprises one or more bacterial lysates from one or more species of a Gram-positive bacterial cell; one or more bacterial lysates from one or more species of a Gram-negative bacterial cell; and a pharmaceutically acceptable carrier and/or excipient, wherein the one or more species of Gram-negative bacterial cell comprises a monophosphoryl Lipid A comprising 5-6 acyl chains such that Gram-negative bacterial lysate comprises the same, and wherein the one or more Gram-positive bacterial lysates and/or the one or more Gram-negative bacterial lysates comprise genomic DNA with a CpG abundance substantially similar to that of a CpG abundance in genomic DNA of a gut bacterium. In various embodiments, the one or more species of Gram-positive bacterial cells comprise lipoteichoic acid (LTA) having a structure substantially similar to the LTA found in F. prausnitzii. In various aspects, the lipoteichoic acid (LTA) comprises an alditolphosphate-containing polymer that is linked via a lipid anchor to the membrane in gram-positive bacteria. In some embodiments, the bacterium comprises a lipoteichoic acid (LTA) selected from any one of Type I, Type II, Type III, Type IV or Type V LTA, as described in Percy et al., (“Lipoteichoic acid synthesis and function in gram-positive bacteria” Annu. Rev. Microbiol. 2014, 68:81-100) the entire disclosure of which is incorporated herein by reference. In various embodiments, the one or more species of Gram-positive bacterial cells is F. prausnitzii. In various aspects, the one or more Gram-positive bacterial lysates and one or more Gram-negative bacterial lysates collectively contain ligands that are capable of binding to toll-like receptor 2, toll-like receptor 4, or NOD2 on a target cell, and such binding being sufficient to activate a cellular response in such target cell.

Any of the compositions provided herein can comprise lysate from at least one of F. prausnitzii, B. thetaiotaomicron, Enterococcus spp, B. vulgatus and/or B. productus. In various aspects, the gut bacterial lysate can comprise lysate from F. prausnitzii and/or B. thetaiotaomicron. In further embodiments, the gut bacterial lysate can comprise lysate from E. faecium, E. faecalis, E. gallinarum, and/or E. hirae. In further embodiments, the gut bacterial lysate can comprise lysate from B. vulgatus and/or B. productus. Hereinafter combinations of these bacteria may be referred to in abbreviated form. For example, lysate prepared from a combination of Faecalibacterium prausnitzii and Bacteroides thetaiotaomicron can be referred to as Fp/Bt or Bt/Fp. Likewise, Bacteroides thetaiotaomicron is commonly referenced as B. thetaiotaomicron or B. theta throughout this disclosure.

In any of the compositions provided herein, synthetic analogues of one or more components found in a gut bacterial lysate may be included. As used herein, the term “synthetic analogue” refers to a component that has a similar structure and function to a reference component (i.e., one obtained from the lysate of one or more species of bacteria). Structural similarity can, in the case of a small molecule like a lipid, involve having the same chemical structure with optionally minor changes that do not impact the function, solubility, or other properties of the molecule. Structural similarity can, in the case of a larger biologic (i.e., a protein or peptide or nucleic acid) involve having the same amino acid or nucleic acid sequence with optionally minor substitutions or alterations that do not affect the properties of the biologic.

### Preparation of the Bacterial Lysates and Components of Bacterial Lysates

The lysates described herein may be prepared by methods known to those of skill in the art. By way of a non-limiting example, lysates may be obtained by one or more rounds of freeze thawing of the bacteria followed by one or more rounds of sonication, centrifugation, and removal of the supernatant (lysate). In another non-limiting example, lysates may be prepared using sonication, e.g., sonication in the presence of beads or other particles.

In some embodiments, two or more bacterial lysates may be combined to provide a composition comprising at least two bacterial lysates.

In some embodiments, one or more components may be isolated from the bacterial lysates or from a combination of one or more bacterial lysates to obtain one or more components of a bacterial lysate. In various embodiments, the one or more components isolated from the bacterial lysates may include lipids, nucleic acids, proteins, peptides or any other biological components derived from a cell. Bacterial lysates may be treated to isolate and, optionally amplify, any fraction of interest using any standard method known in the art (e.g., chromatography, chelation, polymerase replication). As another non-limiting example, the bacterial lysate can be fractionated into polar and nonpolar components and then further sub-fractionated into pure (or mostly pure) fractions of single components thereof. In some embodiments, the isolated component of the bacterial lysate comprises a polar fraction. In some embodiments, the isolated component of the bacterial lysate comprises a lipid (e.g., a Lipid A). In some embodiments, the isolated component of the bacterial lysate comprises a nucleic acid, such as genomic DNA.

As will be appreciated by those of skill in the art, additional components may be added to the bacterial lysates or components of bacterial lysates to increase stability, allow for delivery, etc. Some examples of components that may be added are discuss further herein.

### Preparation of Synthetic Analogues

In various embodiments, preparation of a synthetic analogue based on one or more components isolated from a bacterial lysate may depend on the identity of the synthetic analogue. For example, if the synthetic analogue is based on a small chemical molecule, it can be prepared using standard methods of chemical synthesis. If the synthetic analogue is a larger biologic (i.e., a protein, peptide, or nucleic acid), it can be prepared using standard methods of recombinant expression or chemical (i.e., polymer) synthesis.

### 2. Pharmaceutical Formulations

Any of the compositions comprising the gut bacterial lysate according to the disclosure herein may be formulated as a pharmaceutical formulation or composition. Accordingly, the composition may further comprise a pharmaceutical carrier or excipient. In various embodiments, the composition may be formulated for oral or parenteral administration.

In various embodiments, the pharmaceutical formulation is a liquid formulation comprising one or more components of gut bacteria or synthetic analogue thereof in a phosphate buffered saline solution.

In various embodiments, the liquid formulation can comprise a pH of from about 6.8 to 7.5.

In further embodiments, the liquid formulation can comprise a pH from about 7.35 to about 7.45.

### Pharmaceutically Acceptable Carriers and Excipients

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

In various embodiments, compositions disclosed herein may further comprise one or more pharmaceutically acceptable diluent(s), excipient(s), or carrier(s). As used herein, a pharmaceutically acceptable diluent, excipient, or carrier, refers to a material suitable for administration to a subject without causing undesirable biological effects or interacting in a deleterious manner with any of the components of the composition in which it is contained. Pharmaceutically acceptable diluents, carriers, and excipients can include, but are not limited to, physiological saline, Ringer's solution, phosphate solution or buffer, buffered saline, and other carriers known in the art. Pharmaceutical compositions may also include stabilizers, anti-oxidants, colorants, other medicinal or pharmaceutical agents, carriers, adjuvants, preserving agents, stabilizing agents, wetting agents, emulsifying agents, solution promoters, salts, solubilizers, antifoaming agents, antioxidants, dispersing agents, surfactants, and combinations thereof. Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. Techniques for formulation and administration of drugs may be found in “Remington's Pharmaceutical Sciences,” Mack Publishing Co., Easton, Pa., latest edition, which is incorporated herein by reference.

In various embodiments, pharmaceutical compositions described herein may be formulated in conventional manner using one or more physiologically acceptable carriers comprising excipients and auxiliaries to facilitate processing of genetically modified endothelial progenitor cells into preparations which can be used pharmaceutically. In other embodiments, any of the well-known techniques, carriers, and excipients may be used as suitable and as understood in the art.

In various embodiments, pharmaceutical compositions described herein may be an aqueous suspension comprising one or more polymers as suspending agents. In some embodiments, polymers that may comprise pharmaceutical compositions described herein include: water-soluble polymers such as cellulosic polymers, e.g., hydroxypropyl methylcellulose; water-insoluble polymers such as cross-linked carboxyl-containing polymers; mucoadhesive polymers, selected from, for example, carboxymethylcellulose, carbomer (acrylic acid polymer), poly(methylmethacrylate), polyacrylamide, polycarbophil, acrylic acid/butyl acrylate copolymer, sodium alginate, and dextran; or a combination thereof. In other embodiments, compositions disclosed herein may comprise at least 5%, at least 10%, at least 20%, at least 25%, at least 30%, at least 35%, at least 40%, at least 45%, at least 50% total amount of polymers as suspending agent(s) by total weight of the composition.

In various embodiments, pharmaceutical compositions disclosed herein may comprise a viscous formulation. In some embodiments, viscosity of the composition may be increased by the addition of one or more gelling or thickening agents. In other embodiments, compositions disclosed herein may comprise one or more gelling or thickening agents in an amount to provide a sufficiently viscous formulation to remain on treated tissue. In still other embodiments, compositions disclosed herein may comprise at least 5%, at least 10%, at least 20%, at least 25%, at least 30%, at least 35%, at least 40%, at least 45%, at least 50% total amount of gelling or thickening agent(s) by total weight of the composition. In yet other embodiments, suitable thickening agents can be hydroxypropyl methylcellulose, hydroxyethyl cellulose, polyvinylpyrrolidone, carboxymethyl cellulose, polyvinyl alcohol, sodium chondroitin sulfate, sodium hyaluronate. In other embodiments, viscosity enhancing agents can be acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), Splenda® (dextrose, maltodextrin and sucralose), or combinations thereof. In some embodiments, suitable thickening agent may be carboxymethylcellulose.

In various embodiments, pharmaceutical compositions disclosed herein may comprise additional agents or additives selected from a group including surface-active agents, detergents, solvents, acidifying agents, alkalizing agents, buffering agents, tonicity modifying agents, ionic additives effective to increase the ionic strength of the solution, antimicrobial agents, antibiotic agents, antifungal agents, antioxidants, preservatives, electrolytes, antifoaming agents, oils, stabilizers, enhancing agents, and the like. In some embodiments, pharmaceutical compositions disclosed herein may comprise at least 5%, at least 10%, at least 20%, at least 25%, at least 30%, at least 35%, at least 40%, at least 45%, at least 50% total amount of one or more agents by total weight of the composition. In other embodiments, one or more of these agents may be added to improve the performance, efficacy, safety, shelf-life and/or other property of the muscarinic antagonist composition of the present disclosure. In s embodiments, additives will be biocompatible, and will not be harsh, abrasive, or allergenic.

In various embodiments, pharmaceutical compositions disclosed herein may comprise one or more acidifying agents. As used herein, “acidifying agents” refers to compounds used to provide an acidic medium. Such compounds include, by way of example and without limitation, acetic acid, amino acid, citric acid, fumaric acid and other alpha hydroxy acids, such as hydrochloric acid, ascorbic acid, and nitric acid and others known to those of ordinary skill in the art. In some embodiments, any pharmaceutically acceptable organic or inorganic acid may be used. In other embodiments, compositions disclosed herein may comprise at least 5%, at least 10%, at least 20%, at least 25%, at least 30%, at least 35%, at least 40%, at least 45%, at least 50% total amount of one or more acidifying agents by total weight of the composition.

In various embodiments, pharmaceutical compositions disclosed herein may comprise one or more alkalizing agents. As used herein, “alkalizing agents” are compounds used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, and trolamine and others known to those of ordinary skill in the art. In some embodiments, any pharmaceutically acceptable organic or inorganic base can be used. In other embodiments, compositions disclosed herein may comprise at least 5%, at least 10%, at least 20%, at least 25%, at least 30%, at least 35%, at least 40%, at least 45%, at least 50% total amount of one or more alkalizing agents by total weight of the composition.

In various embodiments, pharmaceutical compositions disclosed herein may comprise one or more antioxidants. As used herein, “antioxidants” are agents that inhibit oxidation and thus can be used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite and other materials known to one of ordinary skill in the art. In some embodiments, compositions disclosed herein may comprise at least 5%, at least 10%, at least 20%, at least 25%, at least 30%, at least 35%, at least 40%, at least 45%, at least 50% total amount of one or more antioxidants by total weight of the composition.

In other embodiments, pharmaceutical compositions disclosed herein may comprise a buffer system. As used herein, a “buffer system” is a composition comprised of one or more buffering agents wherein “buffering agents” are compounds used to resist change in pH upon dilution or addition of acid or alkali. Buffering agents include, by way of example and without limitation, potassium metaphosphate, potassium phosphate, monobasic sodium acetate and sodium citrate anhydrous and dihydrate and other materials known to one of ordinary skill in the art. In some embodiments, any pharmaceutically acceptable organic or inorganic buffer can be used. In another aspect, compositions disclosed herein may comprise at least 5%, at least 10%, at least 20%, at least 25%, at least 30%, at least 35%, at least 40%, at least 45%, at least 50% total amount of one or more buffering agents by total weight of the composition. In other aspects, the amount of one or more buffering agents may depend on the desired pH level of a composition. In some embodiments, pharmaceutical compositions disclosed herein may have a pH of about 6 to about 9. In other embodiments, pharmaceutical compositions disclosed herein may have a pH greater than about 8, greater than about 7.5, greater than about 7, greater than about 6.5, or greater than about 6. In a preferred embodiment, compositions disclosed herein may have a pH greater than about 6.8.

In various embodiments, pharmaceutical compositions disclosed herein may comprise one or more preservatives. As used herein, “preservatives” refers to agents or combination of agents that inhibits, reduces or eliminates bacterial growth in a pharmaceutical dosage form. Non-limiting examples of preservatives include Nipagin, Nipasol, isopropyl alcohol and a combination thereof. In some embodiments, any pharmaceutically acceptable preservative can be used. In other embodiments, pharmaceutical compositions disclosed herein may comprise at least 5%, at least 10%, at least 20%, at least 25%, at least 30%, at least 35%, at least 40%, at least 45%, at least 50% total amount of one or more preservatives by total weight of the composition.

In other embodiments, pharmaceutical compositions disclosed herein may comprise one or more surface-acting reagents or detergents. In some embodiments, surface-acting reagents or detergents may be synthetic, natural, or semi-synthetic. In other embodiments, compositions disclosed herein may comprise anionic detergents, cationic detergents, zwitterionic detergents, ampholytic detergents, amphoteric detergents, nonionic detergents having a steroid skeleton, or a combination thereof. In still other embodiments, pharmaceutical compositions disclosed herein may comprise at least 5%, at least 10%, at least 20%, at least 25%, at least 30%, at least 35%, at least 40%, at least 45%, at least 50% total amount of one or more surface-acting reagents or detergents by total weight of the composition.

In various embodiments, pharmaceutical compositions disclosed herein may comprise one or more stabilizers. As used herein, a “stabilizer” refers to a compound used to stabilize an active agent against physical, chemical, or biochemical process that would otherwise reduce the therapeutic activity of the agent. Suitable stabilizers include, by way of example and without limitation, succinic anhydride, albumin, sialic acid, creatinine, glycine and other amino acids, niacinamide, sodium acetyltryptophonate, zinc oxide, sucrose, glucose, lactose, sorbitol, mannitol, glycerol, polyethylene glycols, sodium caprylate and sodium saccharin and others known to those of ordinary skill in the art. In some embodiments, pharmaceutical compositions disclosed herein may comprise at least 5%, at least 10%, at least 20%, at least 25%, at least 30%, at least 35%, at least 40%, at least 45%, at least 50% total amount of one or more stabilizers by total weight of the composition.

In other embodiments, pharmaceutical compositions disclosed herein may comprise one or more tonicity agents. As used herein, a “tonicity agents” refers to a compound that can be used to adjust the tonicity of the liquid formulation. Suitable tonicity agents include, but are not limited to, glycerin, lactose, mannitol, dextrose, sodium chloride, sodium sulfate, sorbitol, trehalose and others known to those or ordinary skill in the art. Osmolarity in a composition may be expressed in milliosmoles per liter (mOsm/L). Osmolarity may be measured using methods commonly known in the art. In preferred embodiments, a vapor pressure depression method is used to calculate the osmolarity of the compositions disclosed herein. In some embodiments, the amount of one or more tonicity agents comprising a pharmaceutical composition disclosed herein may result in a composition osmolarity of about 150 mOsm/L to about 500 mOsm/L, about 250 mOsm/L to about 500 mOsm/L, about 250 mOsm/L to about 350 mOsm/L, about 280 mOsm/L to about 370 mOsm/L or about 250 mOsm/L to about 320 mOsm/L. In other embodiments, a composition herein may have an osmolality ranging from about 100 mOsm/kg to about 1000 mOsm/kg, from about 200 mOsm/kg to about 800 mOsm/kg, from about 250 mOsm/kg to about 500 mOsm/kg, or from about 250 mOsm/kg to about 320 mOsm/kg, or from about 250 mOsm/kg to about 350 mOsm/kg or from about 280 mOsm/kg to about 320 mOsm/kg. In some embodiments, a pharmaceutical composition described herein has an osmolarity of about 100 mOsm/L to about 1000 mOsm/L, about 200 mOsm/L to about 800 mOsm/L, about 250 mOsm/L to about 500 mOsm/L, about 250 mOsm/L to about 350 mOsm/L, about 250 mOsm/L to about 320 mOsm/L, or about 280 mOsm/L to about 320 mOsm/L. In still other embodiments, pharmaceutical compositions disclosed herein may comprise at least 5%, at least 10%, at least 20%, at least 25%, at least 30%, at least 35%, at least 40%, at least 45%, at least 50% total amount of one or more tonicity modifiers by total weight of the composition.

Suitable routes of administration may, for example, include oral, rectal, transmucosal, intestinal or parenteral delivery, including intramuscular, subcutaneous and intramedullary injections as well as, intravenous, intraperitoneal, intranasal, intrathecal injections. In various embodiments, the route of administration is subcutaneous, oral, intraperitoneal, intrathecal or intravenous.

One may administer the pharmaceutical composition in a local or systemic manner, for example, via local injection of the pharmaceutical composition directly into a tissue region of a patient. In some embodiments, a pharmaceutical composition disclosed herein can be administered parenterally, e.g. intravenous, intraperitoneal, intramuscular, intrathecal, or subcutaneous injection or a combination thereof. In some embodiments, a pharmaceutical composition disclosed herein can administered to the human patient via at least two administration routes. In some examples, the combination of administration routes involves at least two administration techniques selected from the group consisting of subcutaneous, intravenous, intraperitoneal, and oral administration. For example, a combination of administration routes may comprise subcutaneous injection and intravenous injection; intrathecal injection and intravenous injection; intrathecal injection and subcutaneous injection; and intra-tumoral injection and intravenous injection.

Pharmaceutical compositions of the present disclosure may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water-based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol, or dextran. Optionally, the suspension may also contain suitable stabilizers or agents which increase the solubility of the active ingredients to allow for the preparation of highly concentrated solutions.

In some embodiments, the pharmaceutical composition is formulated for subcutaneous administration. Compositions formulated for this route may further comprise hyaluronidase. Additional excipients suitable for preparing subcutaneous compositions are provided in Turner et al., (“Challenges and opportunities for the subcutaneous delivery of therapeutic proteins” Journal of Pharmaceutical Sciences. Volume 107, Issue 5, May 2018, Pages 1247-1260) which is incorporated herein by reference in its entirety.

Alternatively, the active ingredient may be in powder form for constitution with a suitable vehicle, e.g., sterile, pyrogen-free water-based solution, before use.

Pharmaceutical compositions suitable for use in context of the present disclosure include compositions wherein the active ingredients (e.g., bacterial lysates and/or components thereof) are contained in an amount effective to achieve the intended purpose. In some embodiments, a therapeutically effective amount means an amount of active ingredients effective to prevent, slow, alleviate or ameliorate symptoms of a cancer or prolong the survival of the subject being treated.

Determination of a therapeutically effective amount is well within the capability of those skilled in the art, especially in light of the detailed disclosure provided herein.

For any preparation used in the methods of the present disclosure, the therapeutically effective amount or dose can be estimated initially from in vitro and cell culture assays and or screening platforms disclosed herein. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1, incorporated by reference in its entirety).

Dosage amount and interval may be adjusted individually to brain or blood levels of the active ingredient are sufficient to induce or suppress the biological effect (minimal effective concentration, MEC). The MEC will vary for each preparation but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc. Effective doses may be extrapolated from dose-responsive curves derived from in vitro or in vivo test systems

### 3. Methods and Uses

The compositions and formulations as described herein may be used to treat various cancers. Accordingly, in various aspects, a method is provided for the treatment of cancer in a subject in need thereof, the method comprising (a) administering a therapeutically effective amount of any of the compositions provided herein (compositions comprising one or more bacterial lysates, components of bacterial lysates, or synthetic analogs thereof, etc.) to the subject. In some embodiments, the method further comprises (b) administering a therapeutically effective amount of a cancer treatment to the subject.

In some embodiments, step (a) comprises administering orally or parenterally administering a therapeutically effective amount of at least one gut bacterial lysate, at least one or more component of at least one gut bacterial lysate, or synthetic analog thereof to the subject in need thereof. In some embodiments, step (a) comprises administering orally or parenterally administering a therapeutically effective amount of at least one gut bacterial lysate or synthetic analog thereof to the subject in need thereof. In some embodiments, step (a) comprises administering orally or parenterally administering a therapeutically effective amount of at least one or more component of at least one gut bacterial lysate or synthetic analog thereof to the subject in need thereof. In some embodiments, the gut bacterial lysate is a pharmaceutical composition and further comprises at least one pharmaceutically acceptable carrier and/or excipient.

In some embodiments, step (a) comprises administering orally or parenterally administering a therapeutically effective amount of at least one gut bacterial lysate to the subject in need thereof, wherein the at least one gut bacterial lysate comprises one or more bacterial lysates from one or more species of a Gram-positive bacterial cell and one or more bacterial lysates from one or more species of Gram-negative bacterial cell. In some embodiments, the one or more species of a Gram-negative bacterial cell is B. thetaiotaomicron, B. vulgatus, or any combination thereof. In some embodiments, the one or more species of a Gram-positive bacterial cell is E. faecium, E. faecalis, E. gallinarum, E. hirae, or any combination thereof. In some embodiments, the one or more species of Gram-negative bacterial cell comprises a monophosphoryl Lipid A comprising 5-6 acyl chains such that Gram-negative bacterial lysate comprises the same, and wherein the one or more Gram-positive bacterial lysates and/or the one or more Gram-negative bacterial lysates comprise genomic DNA with a CpG abundance substantially similar to that of a CpG abundance in genomic DNA of a gut bacterium. In some embodiments, the one or more species of Gram-positive bacterial cells comprise lipoteichoic acid (LTA) having a structure substantially similar to the LTA found in F. prausnitzii. In some embodiments, the one or more Gram-positive bacterial lysates and one or more Gram-negative bacterial lysates collectively contain ligands that are capable of binding to toll-like receptor 2, toll-like receptor 4, and NOD2 on a target cell, and such binding being sufficient to activate a cellular response in such target cell. In some embodiments, the gut bacterial lysate is a pharmaceutical composition and further comprises at least one pharmaceutically acceptable carrier and/or excipient.

In embodiments, step (a) comprises administering orally or parenterally administering a therapeutically effective amount of a composition to the subject in need thereof, wherein the composition comprises one or more components from one or more bacterial lysates from one or more species of a Gram-positive bacterial cell and one or more components from one or more bacterial lysates from one or more species of a Gram-negative bacterial cell. In some embodiments, the one or more species of a Gram-negative bacterial cell is B. thetaiotaomicron, B. vulgatus, or any combination thereof. In some embodiments, the one or more species of a Gram-positive bacterial cell is E. faecium, E. faecalis, E. gallinarum, E. hirae, or any combination thereof. In some embodiments the one or more species of Gram-negative bacterial cell comprises a monophosphoryl Lipid A comprising 5-6 acyl chains such that Gram-negative bacterial lysate comprises the same, and wherein the one or more Gram-positive bacterial lysates and/or the one or more Gram-negative bacterial lysates comprise genomic DNA with a CpG abundance substantially similar to that of a CpG abundance in genomic DNA of a gut bacterium. In some embodiments, the one or more species of Gram-positive bacterial cells comprise lipoteichoic acid (LTA) having a structure substantially similar to the LTA found in F. prausnitzii. In some embodiments, the one or more Gram-positive bacterial lysates and one or more Gram-negative bacterial lysates collectively contain ligands that are capable of binding to toll-like receptor 2, toll-like receptor 4, and NOD2 on a target cell, and such binding being sufficient to activate a cellular response in such target cell. In some embodiments, the composition is a pharmaceutical composition and further comprises at least one pharmaceutically acceptable carrier and/or excipient.

In embodiments, step (a) comprises administering orally or parenterally administering a therapeutically effective amount of at least one gut bacterial lysate to the subject in need thereof, wherein the at least one gut bacterial lysate comprises one or more bacterial lysates from one or more species of bacteria having genomic DNA with a CpG abundance substantially similar to a CpG abundance in genomic DNA of a gut bacterium. In some embodiments, the species of bacteria is F. prausnitzi, B. thetaiotaomicron, B. producta, B. vulgatus, or any combination thereof. In some embodiments, the gut bacterial lysate is a pharmaceutical composition and further comprises at least one pharmaceutically acceptable carrier and/or excipient.

In embodiments, step (a) comprises administering orally or parenterally administering a therapeutically effective amount of at least one gut bacterial lysate to the subject in need thereof, wherein the at least one gut bacterial lysate comprises one or more bacterial lysate from a species of bacteria that comprises a Lipid A structure substantially similar to a Lipid A in B. thetaiotaomicron. In some embodiments, the Lipid A structure comprises a monophosphoryl Lipid A comprising 5-6 acyl chains. In some embodiments, the gut bacterial lysate is a pharmaceutical composition and further comprises at least one pharmaceutically acceptable carrier and/or excipient.

In some embodiments, step (a) and (b) are administered at least partially simultaneously. In some embodiments, step (a) is administered alongside with step (b). The terms “alongside with”, “in combination with”, and “co-administration” are not limited to the administration of agents at exactly the same time. Instead, it is meant that the lysate composition disclosed herein, and another cancer treatment are administered in a sequence and within a time interval such that they may act together to provide a benefit. In some embodiments, the benefit is increased versus treatment with only either the disclosed composition or the cancer treatment. In some embodiments, the two agents are administered at a time where both agents are active in the subject at the same time. Such agents are suitably present in combination in amounts that are effective for the purpose intended. The skilled medical practitioner can determine empirically, or by considering the pharmacokinetics and modes of action of the agents, the appropriate dose or doses of each agent, as well as the appropriate timings and methods of administration.

### Step (a)

Any of the pharmaceutical compositions provided herein may be administered in step (a) of the methods herein.

In various embodiments, step (a) comprises orally administering the pharmaceutical composition to the subject. In some embodiments, step (a) comprises parenterally administering the pharmaceutical composition to the subject. In various aspects, parenteral administration of the composition in step (a) can comprise intravenous, intraperitoneal, intramuscular, intrathecal, or subcutaneous administration.

In some embodiments, the composition in step (a) is administered subcutaneously. In further embodiments, step (a) comprises subcutaneously administering the pharmaceutical composition ipsilaterally to a tumor in the subject. In other embodiments, step (a) comprises subcutaneously administering the pharmaceutical composition contralaterally to a tumor in the subject.

In other embodiments, parenteral administration of the composition in step (a) can be intravenous. In some embodiments, step (a) comprises administering the pharmaceutical composition using at least two administration techniques selected from the group consisting of subcutaneous, intravenous, intraperitoneal, and oral administration. For example, in some embodiments, step (a) comprises administering the pharmaceutical composition (i.e., the gut microbiota lysate) both intravenously and subcutaneously close to a draining lymph node of a metastasis.

In any of the methods described herein, the pharmaceutical composition is administered orally or parenterally. In various embodiments, parenteral administration of the pharmaceutical composition is intravenous, intramuscular, intrathecal, intraperitoneal or subcutaneous. In various embodiments, the parenteral administration of the composition is subcutaneous. In some instances, the parenteral administration of the composition to the subject may be an adjunct therapy to the cancer treatment. In some cases, the parenteral administration of the composition to the subject may be part of a combination therapy in which the parenteral administration of the composition to the subject is administered at substantially the same time or during the administration of the cancer treatment. In at least some instances, the method may be a combination method for the treatment of cancer that includes administering to a subject in need thereof a therapeutic combination comprising: (a) parenteral administration of a composition disclosed herein to the subject and (b) administration of a cancer treatment to the subject.

The subcutaneous route for delivery provides some unexpected advantages for the delivery of the composition to the tumor site. Without being bound to any theory, it is believed that administering the composition (e.g., bacterial lysates) subcutaneously may reduce toxicity and also allows for the bacterial lysate and/or components of the bacterial lysate to be directed/shunted towards the nearest secondary lymphoid organ (lymph node) and thus the bacterial pathogen-associated molecular patterns (PAMPS) are “delivered” to the immune cells that it can activate. If the lymph node is the tumor draining lymph node (lymph node most adjacent to the tumor), then this facilitates tumor killing as the primed/activated T cells can then go to the tumor and kill cancer cells. Accordingly, in various embodiments, the subcutaneous administration of the composition occurs ipsilaterally to a tumor in the subject. In various embodiments, the subcutaneous administration of the composition occurs contralaterally to a tumor in the subject.

In various embodiments, the administration of the composition disclosed herein can be altered based on clinical circumstances. If, for example, local control of a primary tumor is desired, then the composition (e.g., bacterial lysate) can be administered subcutaneously close to a draining lymph node adjacent (or most adjacent) to the tumor. Alternatively, to treat a metastatic or potentially metastatic tumor, the composition can be administered subcutaneously closes to the draining lymph node of the metastasis (e.g., axillary lymph node for lung metastases) and also administered intravenously to allow for delivery to other secondary lymphoid organs. As another example, to treat a brain tumor, the composition can be administered intrathecally such as into cerebrospinal fluid. Various combinations of subcutaneous, intravenous, intraperitoneal, intrathecal and oral administration can be envisioned by one of ordinary skill in the art, in view of the specific clinical presentation of the subject.

In various embodiments, the disclosed composition may be administered in a dose from about 0.0005 to 10 mg/kg. For example, in some embodiments, the composition may be administered in a dose from about 0.3 to 9.8 mg/kg. In some embodiments, the composition may be administered monthly for 3 to 12 months, 3 to 10 months, or 3 to 6 months. In some embodiments, the composition may be administered weekly (e.g., once every week for 1, 2, 3, 4, 5, 6, 7, 8, 9, 10, 11, 12 or more weeks). In some embodiments, the composition may be administered every other day, or every day (e.g., for 1, 2, 3 or 4 months). The dosage may be further divided so that more than one dose is provided per day. Suitable dosing and timing thereof may be determined as appropriate as understood in the art.

### Step (b)

The methods provided herein can further comprise (b) administering any cancer treatment to the subject. In some embodiments, the cancer treatment comprises immunotherapy, chemotherapy, hormone therapy, radiation therapy, stem cell transplant, surgery, and/or any combination thereof.

In some embodiments, the cancer treatment comprises immunotherapy. In various embodiments, the cancer immunotherapy comprises (a) administering an immune checkpoint inhibitor therapy (ICT), (b) administering modified immune cells to the subject, (c) administering a bi-specific antibody to the subject, or any combination thereof. Immune checkpoint inhibitor therapy (ICT) involves administering small molecule agents and/or biologics (i.e., antibodies or proteins) that target checkpoint receptors on immune cells, releasing native immune suppression and increasing the immune response to tumors. The ICT administered in the methods of the instant disclosure can comprise, in various embodiments, an anti-PD-1 an anti-PD-L1 therapy, an anti-CTLA-4, an anti-LAG-3, an anti-PD-1H, therapy or any combination thereof. Also contemplated are antibodies or therapies targeting lymphocyte activation gene-3 (LAG-3), B and T lymphocyte attenuator (BTLA), programmed death-1 homolog (PD-1H), T-cell immunoglobulin and immunoreceptor tyrosine-based inhibitory motif domain (TIM-3)/carcinoembryonic antigen cell adhesion molecule 1 (CEACAM1), and the poliovirus receptor (PVR)-like receptors or others described in Torphy et al., (“Newly Emerging Immune Checkpoints: Promises for Future Cancer Therapy” Int J Mol Sci. 2017 Dec. 6; 18(12):2642) which are incorporated by reference herein in their entirety. In some embodiments, the ICT administered in step (b) can target a “next generation” immunotherapy target such as: LAG3, TIGIT, TIM3, VISTA, B7-H3, Siglec-15, 4-1BB, IDO1, GITR, BTLA, PD-1H, CD96, CD112R, CD200R or any combination thereof. Additionally, intracellular molecules with ubiquitin ligase activity such as CISH and CBLB may be considered immune checkpoint targets and antibodies or small molecules that inhibit them are contemplated for use in the disclosed methods.

In some embodiments, the method comprises administering an anti-PD-1 therapy or an anti-PD-L1 therapy. Both the anti-PD-1 therapy and anti-PD-L1 therapy may be selected from FDA approved or experimental therapies as described in Table C. In various embodiments, the anti-PD-1 therapy comprises pembrolizumab, nivolumab, cemiplimab, spartalizumab, sintilimab, tislelizumab, toripalimab, dostalimab. or any combination thereof. In various embodiments, the anti-PD-L1 therapy comprises atezolizumab, avelumab, durvalumab, KN035, AUNP12 or any combination thereof. In other embodiments, the method comprises administering an anti-CTLA-4 therapy such as ipilimumab. In still other embodiments, the method comprises administering both an anti-CTLA-4 therapy and an anti-PD-1 or an anti-PD-L1 therapy. Accordingly, in various embodiments, the method can comprise administering ipilimumab and at least one of pembrolizumab, nivolumab, cemiplimab, atezolizumab, avelumab, durvalumab, spartalizumab, sintilimab, tislelizumab, toripalimab, dostalimab, KN035, or AUNP12.

TABLE C
Exemplary anti-PD-1 and anti-PD-L1 Therapies

| | |
|---|---|
| FDA-approved a-PD-1 | Pembrolizumab, nivolumab, cemiplimab |
| therapies | |
| Experimental PD-1 | Spartalizumab, Sintilimab, Tislelizumab, |
| inhibitors | Toripalimab, Dostalimab |
| FDA-approved a-PD-L1 | Atezolizumab, Avelumab, Durvalumab |
| therapies | |
| Experimental PD-L1 | KN035 - subcutaneous PD-L1 nanobody ¹ |
| inhibitors | AUNP12 - peptide inhibitor of PD-1/PD-L1 ² |

¹ Zhang et al., “Structural basis of a novel PD-L1 nanobody for immune checkpoint blockade” Cell Discov. 2017; 3: 17004.
² Juneja et al., “PD-L1 on tumor cells is sufficient for immune evasion in immunogenic tumors and inhibits CD8 T cell cytotoxicity” J Exp Med. Apr. 3, 2017; 214(4): 895-904.

Another form of immunotherapy involves isolating immune cells from a patient or a donor, genetically modifying them to increase their selectivity to a tumor and reinfusing them into a subject. The modified immune cells can comprise modified dendritic cells (DCs), modified natural killer (NK) cells and/or CAR-T cells. CAR-T cells are T cells that have been engineered to express a modified chimeric antigen receptor (CAR) that has a dual ability to bind to a tumor specific antigen and stimulate a cytotoxic immune response. Accordingly, in various embodiments, the methods provided herein comprise administering to a subject a modified immune cell such as a modified dendritic cell, a modified NK cell and/or a CAR-T cell. In various embodiments, the modified immune cell may have selective affinity (or may target) an antigen on a tumor. In various embodiments, the antigen may be an antigen specific for a carcinoma, a sarcoma, or a hematologic cancer (i.e., leukemias, lymphomas, multiple myeloma etc). In various embodiments, the antigen may be an antigen specific for any of the following cancers: squamous cell head and neck cancer, colon cancer, colorectal cancer, Acute myeloid leukemia (AML), Chronic myeloid leukemia (CML) Acute lymphoblastic leukemia (ALL), Merkel cell carcinoma, cutaneous squamous cell carcinoma, hepatocellular carcinoma, advanced renal cell carcinoma, microsatellite instability-high (MSI-H) or mismatch repair deficient (dMMR) cancers, cervical cancer, small cell lung cancer, non-small cell lung cancer, triple-negative breast cancer, gastric and gastroesophageal junction (GEJ) carcinoma, classical Hodgkin lymphoma, primary mediastinal B-cell lymphoma (PMBCL), and locally advanced or metastatic urothelial cancer. In some instances, the antigen is specific for a melanoma or metastatic melanoma. In some instances, the antigen is specific for a colon cancer or colorectal cancer. In some instances, the antigen is specific for acute B-cell lymphoblastic leukemia. In some instances, the antigen is specific for non-small cell lung cancer.

In some embodiments, the CAR-T cells are HLA-matched CAR-T cells (e.g., CART CD19 cells).

Another form of immunotherapy comprises administering bispecific antibodies to a subject. Bispecific antibodies contemplated for use with the methods described herein have two different antigen binding sites. In some embodiments, the two antigen binding sites may target an immune cell (e.g., a T-cell), a tumor, a radiotherapeutic (e.g., a radioactive payload), a signaling molecule or any combination thereof. In some embodiments, the bispecific antibody may have a tumor binding site. For example, in some embodiments, the bispecific antibody may have an immune cell binding site and a tumor or tumor cell binding site. In other embodiments, the bispecific antibody may have a binding site with affinity for a pharmaceutical (e.g., a radioactive pharmaceutical, a chemotherapeutic, a nanoparticle etc). In some embodiments, the bispecific antibody may comprise more than one T cell activating domain, such that application of the bispecific antibody to the T cell activates it. In various embodiments, the T cell activating component can target CD3. In various embodiments, the tumor specific component can target any tumor specific antigen, such as an antigen specific for any of the cancers listed above. In various embodiments, the tumor specific antigen can comprise CD19, CLL-1 or BCMA. Additional information about bispecific antibodies and their applications in cancer treatment is provided in Suurs et al., (“A review of bispecific antibodies and antibody constructs in oncology and clinical challenges” Pharmacology and Therapeutics 201 (2019) 103-119), the entire contents of which are incorporated herein by reference. In some embodiments, the bispecific antibody can comprise one or more of the following (antibody targets are provided in parentheses): blinatumomab (CD19×CD3), Catumaxomab (EpCAM×CD3), MEHD7945A/Duligotuzumab (EGFR×HER3), AFM13 (CD30×CD16A), AMG110 (EpCAM×CD3), AMG211 (CEA×CD3), 81836880 (VEGF×Ang-2), BIS-1 (EpCAM×CD3), CD20Bi (CD20×CD3), DT2219 (CD19×CD22), EGFRBi (EGFR×CD3), EGFR-nanocell-paclitaxel, EGFR-nanocell-doxorubicin, F6-734/hMN14-734 (CEA×DTPA), FBTA05 (CD20×CD3), HER2Bi (HER2×CD3), IMCgp100 (gp100×CD3), IMCgp100 (gp100×CD3), LYS164530 (MET×EGFR), MCL-128 (HER2×HER3), MDX-447 (EGFR×CD64), MM-111 (HER2×HER3), MM-141 (IGF-1R×HER3), OMP-305B83 (DLL4×VEGF), RG7802/RO695688 (CEA×CD3), RO6874813 (FAP×DR5), TargoMIRs (EGFR×EDV-miR16), TF2+IMP288 (CEA×IMP288), Vanucizumab (Ang-2×VEGF-A), ZW25 (HER2×HER2), or any combination thereof.

The methods of immunotherapy that may be administered alongside the bacterial cell lysate compositions are not limited to immune checkpoint inhibitors, modified immune cells or bispecific antibodies as described above. Any effective therapy that has been shown to enhance an anti-tumor immune response may be used.

In some embodiments, the cancer treatment comprises radiation therapy. in various aspects, the radiation therapy comprises 3D conformal radiation therapy, Intensity-modulated radiation therapy (IMRT), Volumetric modulated radiation therapy (VMAT), Image-guided radiation therapy (IGRT), Stereotactic radiosurgery (SRS), Brachytherapy, Superficial x-ray radiation therapy (SXRT, Intraoperative radiation therapy (IORT) or any combination thereof. Additional radiation therapies are provided in Shiao et al., (“Commensal bacteria and fungi differentially regulate tumor responses to radiation therapy” Cancer Cell. 2021 Jul. 29; 51535-6108(21)00379-2) and Guo et al., (“Multi-omics analyses of radiation survivors identify radioprotective microbes and metabolites” Science. 2020 Oct. 30; 370(6516)), the entire disclosure of both are incorporated herein by reference in their entirety.

In some embodiments, the cancer treatment comprises chemotherapy. In some embodiments, the chemotherapy comprises paclitaxel, doxorubicin, carboplatin, cyclophosphamide, daunorubicin, doxorubicin, epirubicin, cyclophosphamide, or any combination thereof. In some embodiments, the chemotherapy comprises cyclophosphamide. Cyclophosphamide is considered a conventional chemotherapeutic agent and the primary mode of action is as an alkylating agent. Its cytotoxic effect is mainly due to cross-linking of strands of DNA and RNA, and to inhibition of protein synthesis of tumor cells. Without being bound by theory, cyclophosphamide may induce translocation of certain gut bacterial into the lymph system and administering the compositions herein may augment and enhance this effect.

In any of the methods herein, any composition described herein may be administered alongside the cancer treatment. In any of the methods herein, any compositions described herein may be administered at the same time as the cancer treatment. In any of the methods herein, any compositions described herein may be administered after the cancer treatment. In some embodiments, certain bacterial cell lysates may be more beneficial when combined with a certain type of cancer treatment.

In some embodiments, the method of treating a cancer in a subject in need thereof can comprise administering to the subject (a) a composition comprising a bacterial lysate from Enterococcus sp. and (b) one or more modified immune cells (e.g., modified NK cells or CAR-T cells). In some embodiments, the Enterococcus sp. comprises E. faecium, E. faecalis, E. gallinarum, and/or E. hirae.

In some embodiments, the method of treating a cancer in a subject in need thereof can comprise administering to the subject (a) a composition comprising one or more one or more components of a lysate from Enterococcus sp and (b) one or more modified immune cells (e.g., modified NK cells or CAR-T cells). In some embodiments, the Enterococcus sp. comprises E. faecium, E. faecalis, E. gallinarum, and/or E. hirae.

In some embodiments, the method can comprise administering to a subject in need thereof (a) a composition comprising a bacterial lysate from F. prausnitzii and/or B. thetaiotaomicron; and (b) an immune checkpoint inhibitor or blocker (ICB). In various embodiments, the immune checkpoint blocker comprises an anti-PD-1 therapy, an anti-PD-L1 therapy, an anti-CTLA-4 therapy or a combination thereof. In some embodiments, the immune checkpoint blocker comprises (a) an anti-PD-1 therapy (e.g., pembrolizumab, nivolumab, cemiplimab), or an anti-PD-L1 therapy (e.g., atezolizumab, avelumab, or durvalumab) and (b) an anti-CTLA-4 therapy (e.g., ipilimumab).

In some embodiments, the method can comprise administering to a subject in need thereof (a) a composition comprising one or more components from a bacterial lysate from F. prausnitzii and/or B. thetaiotaomicron; and (b) an immune checkpoint inhibitor or blocker (ICB). In various embodiments, the immune checkpoint blocker comprises an anti-PD-1 therapy, an anti-PD-L1 therapy, an anti-CTLA-4 therapy or a combination thereof. In some embodiments, the immune checkpoint blocker comprises (a) an anti-PD-1 therapy (e.g., pembrolizumab, nivolumab, cemiplimab), or an anti-PD-L1 therapy (e.g., atezolizumab, avelumab, or durvalumab) and (b) an anti-CTLA-4 therapy (e.g., ipilimumab).

In some embodiments, the method can comprise administering to a subject in need thereof (a) a bispecific antibody and (b) a composition comprising one or more bacterial lysates from F. prausnitzii, B. thetaiotaomicron, B. vulgatus and/or B. productus.

In some embodiments, the method can comprise administering to a subject in need thereof (a) a bispecific antibody and (b) a composition comprising one or more components from a bacterial lysate from F. prausnitzii, B. thetaiotaomicron, B. vulgatus and/or B. productus.

In some embodiments, the method can comprise administering to a subject in need thereof (a) a bispecific antibody and (b) a composition comprising one or more bacterial lysates from Enterococcus sp.

In some embodiments, the method can comprise administering to a subject in need thereof (a) a bispecific antibody and (b) a composition comprising one or more components from a bacterial lysate from Enterococcus sp.

In any of the methods provided herein the cancer treatment may be administered according to methods known in the art. In various embodiments, the cancer treatment is immunotherapy and is administered parenterally. In various embodiments, the cancer treatment is immunotherapy and is administered intravenously or intraperitoneally.

Administration of any of the immunotherapies herein may proceed according to doses and dosing schedules known in the art. In some embodiments, administering a gut microbial lysate composition as provided herein may, advantageously, reduce a therapeutically effective dose of an immunotherapy.

As a non-limiting example, intravenous administration of ipilimumab may include administration of a 3 mg/kg dose every 3 weeks for four doses. Intravenous administration of nivolumab may include, in at least some instances, administration of a 1 mg/kg dose every 3 weeks for four doses. In other instances, intravenous administration of nivolumab may include administration of a 240 mg dose every 2 weeks. Intravenous administration of pembrolizumab may, at least in some instances, administration of a 2 mg/kg dose every 3 weeks for four doses. Ipilimumab, nivolumab, and pembrolizumab may be administered alone or in combination. For example, in some instances, ICT may include intravenous administration of nivolumab at 1 mg/kg with ipilimumab 3 mg/kg every 3 weeks for four doses followed by intravenous administration of nivolumab alone at 240 mg every 2 weeks.

In some embodiments, the administration of modified immune cells can include a single infusion of a bolus of modified immune cells (e.g., CAR-T cells). In some embodiments, administration of modified immune cells can comprise more than one infusion (e.g., in the case of a relapse).

In some embodiments, the administration of bispecific antibodies can include administration of 0.01 to 10.0 mg/kg body weight in a weekly infusion for four weeks. In some embodiments, the administration of bispecific antibodies can include administering 40-1000 mg every three weeks, or 40-180 mg every week. Additional dosing schedules for suitable bispecific antibodies are provided in Suurs et al., (A review of bispecific antibodies and antibody constructs in oncology and clinical challenges” Pharmacology and Therapeutics 201 (2019): 103-119) which is incorporated herein by reference in its entirety.

The methods provided herein all are directed to treating cancer in a subject in need thereof. The subject may be a mammal or a human.

In various embodiments, the cancer that is treated comprises a solid tumor cancer or a blood cancer. In various embodiments, the cancer comprises a carcinoma, a sarcoma, or a hematologic cancer (i.e., leukemias, lymphomas, multiple myeloma etc). In some embodiments, the presently disclosed compositions and methods may be used to treat squamous cell head and neck cancer, colon cancer, colorectal cancer, Acute myeloid leukemia (AML), Chronic myeloid leukemia (CML) Acute lymphoblastic leukemia (ALL), Merkel cell carcinoma, cutaneous squamous cell carcinoma, hepatocellular carcinoma, advanced renal cell carcinoma, microsatellite instability-high (MSI-H) or mismatch repair deficient (dMMR) cancers, cervical cancer, small cell lung cancer, non-small cell lung cancer, triple-negative breast cancer, gastric and gastroesophageal junction (GEJ) carcinoma, classical Hodgkin lymphoma, primary mediastinal B-cell lymphoma (PMBCL), and locally advanced or metastatic urothelial cancer. In some instances, the cancer that is treated may be melanoma or metastatic melanoma. In some instances, the cancer that is treated may be colon cancer or colorectal cancer. In some instances, the cancer that is treated may be acute B-cell lymphoblastic leukemia. In some instances, the cancer that is treated may be non-small cell lung cancer.

### 4. Kits

Any composition provided herein may be particularly suited for the treatment of a cancer in a subject. The subject may be a mammalian or human subject. The compositions may also be particularly suited for use as an adjunct therapy or a combination therapy with an immunotherapy such as immune checkpoint inhibitor therapy (ICT), modified immune cells, bispecific antibodies, or any combination thereof.

Accordingly, a kit is provided for use in the treatment of a cancer in a subject. The kit can comprise (i) one or more gut bacterial lysates in a composition formulated for oral or parenteral administration such as, for example, pharmaceutical compositions as described herein (including compositions comprising one or more components of a bacterial lysate and/or synthetic analogues of one or more bacterial lysate component) and (ii) compositions for a cancer treatment. In some embodiments, the compositions for a cancer treatment are immunotherapy treatments. Accordingly, in some embodiments, the (ii) compositions for a cancer treatment comprise (a) one or more compositions suitable for use in immune checkpoint inhibitor therapy (ICT); (b) one or more compositions suitable for immune cell transfer therapy, or (c) a bispecific antibody. The one or more compositions suitable for use in ICT may be, for example, selected from the group consisting of ipilimumab, nivolumab, pembrolizumab, cemiplimab, atezolizumab, avelumab, durvalumab, Spartalizumab, Sintilimab, Tislelizumab, Toripalimab, Dostalimab, KN035, AUNP12 and any combination thereof. The one or more compositions suitable for immune cell transfer therapy can comprise modified NK cells or CAR-T cells.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1: Lysates from B. thetaiotaomicron (Bt) and F. prausnitzii (Fp) Improve Immunotherapy Outcomes in Melanoma Mouse Model

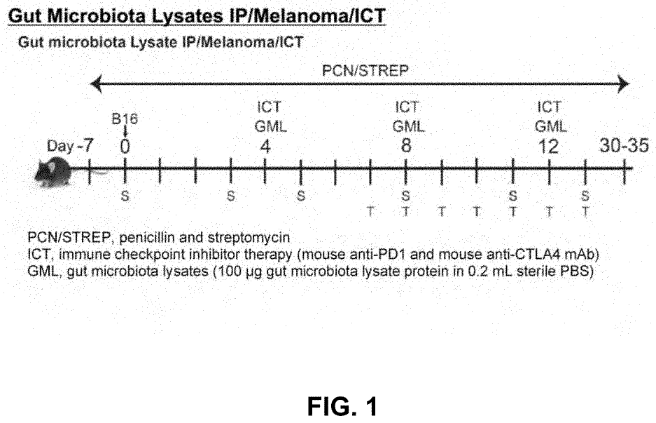

A preclinical melanoma model (immunocompetent C57BL/6 mice with B16-F10 melanoma) was used to investigate the effect of administration of gut microbiota lysates (100 ug as determined by protein concentration using a BCA assay) in combination with ICT therapy in a mouse melanoma model, as shown in FIG. 1. Mice with melanoma (1×10⁵ B16-F10 tumor cells injected subcutaneously in the right flank) were treated with penicillin and streptomycin in the drinking water to significantly deplete gut microbiota and immune checkpoint inhibitor therapy (200 μg mouse anti-PD-1 and 200 μg mouse anti-CTLA4 mAb). Some mice with melanoma were additional treated with gut microbiota lysates (100 μg gut microbiota lysate protein in 0.2 mL sterile PBS). Antibiotics (penicillin/streptomycin) were maintained throughout the duration of the experiment. Loss of survival was defined as death (with moribund mice being euthanized) or when tumor diameter ≥2 cm in any dimension.

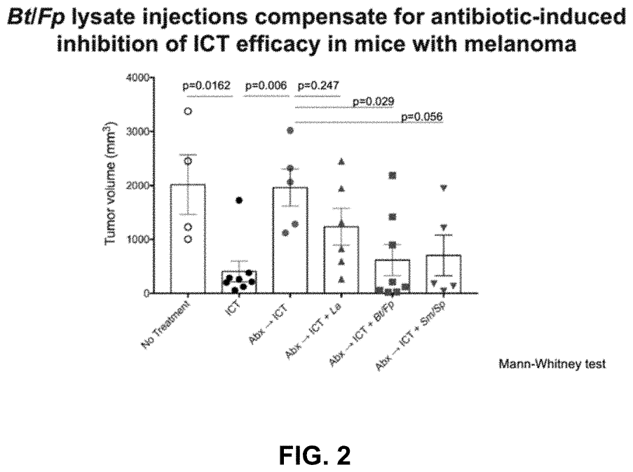

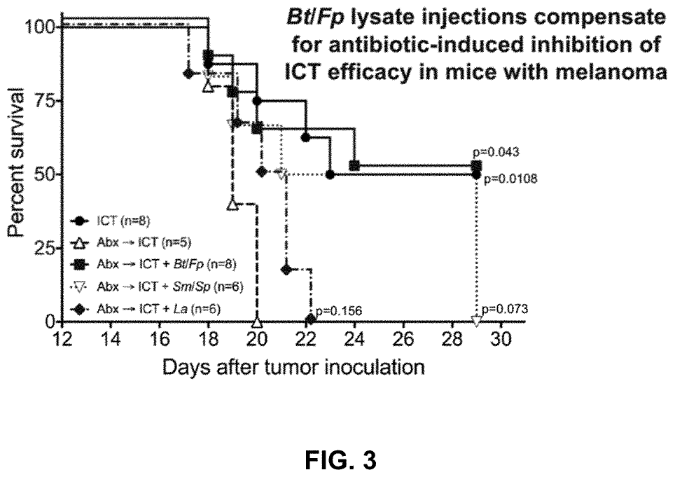

Mice with melanoma who were treated with combination ICT and a gut microbiota lysate composition comprising B. thetaiotaomicron (Bt) and F. prausnitzii (Fp) had smaller tumor growth and increased length of survival compared to mice who were treated with Lactobacillus acidophilus (La), a probiotic commonly found in yogurt, as shown in FIG. 2. As shown in FIG. 3, experiments in which gut microbiota lysates were administered via intraperitoneal injection, it was surprisingly found that lysates of the Gram-negative bacteria B. thetaiotaomicron (Bt) and/or the Gram-positive bacteria F. prausnitzii (Fp) were able to inhibit tumor growth and increased length of survival of mice with melanoma whereas the lysates of Lactobacillus acidophilus did not. These data suggest that administration of bacterial constituents or ligands increases efficacy of ICT against melanoma and can be used as an adjunctive therapy to ICT to optimize therapeutic efficacy.

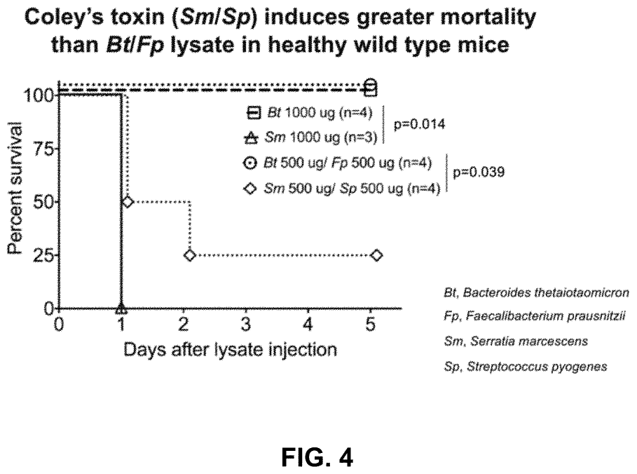

FIG. 4 depicts comparison data showing that Coley's Toxin, a mixture of a pathogenic Gram-negative bacteria and pathogenic Gram-positive bacteria, induces greater mortality than administration of gut microbiota lysates in healthy wild type mice. Specifically, as shown in FIG. 4, injection of high doses (1000 μg) of B. thetaiotaomicron (Bt) and/or F. prausnitzii (Fp) is well-tolerated and safe in mice, whereas injection with Coley's toxin (Serratia marcescens (Sm) and/or Streptococcus pyogenes (Sp) results in 75-100% mortality.

Additionally, data shows that injection with high doses (1000 μg) of lysates of B. thetaiotaomicron and/or F. prausnitzii is well-tolerated and safe in mice. The presently disclosed compositions and methods utilize lysates of dead gut microbiota and therefore have advantages over techniques that use live cells which are often accompanied by potentially adverse effects with introducing new bacteria into the subject. Additionally, the difficulties associated with the need for causing new live bacteria to colonize and take hold in the gut of a subject are avoided by the presently disclosed compositions and techniques.

### Example 2: Gut Microbiota Compositions Between Patients with Different Immunotherapy Outcomes

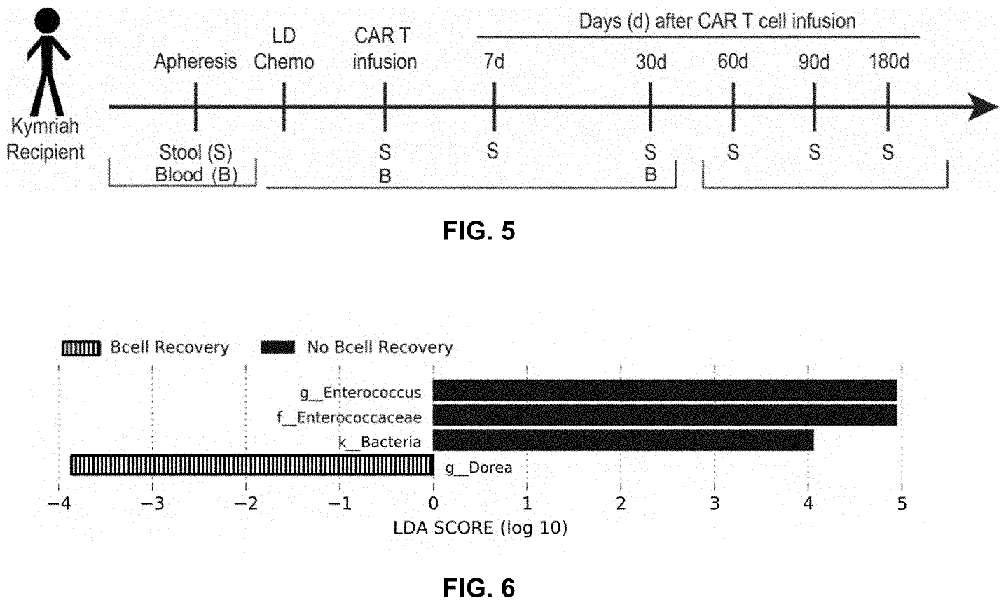

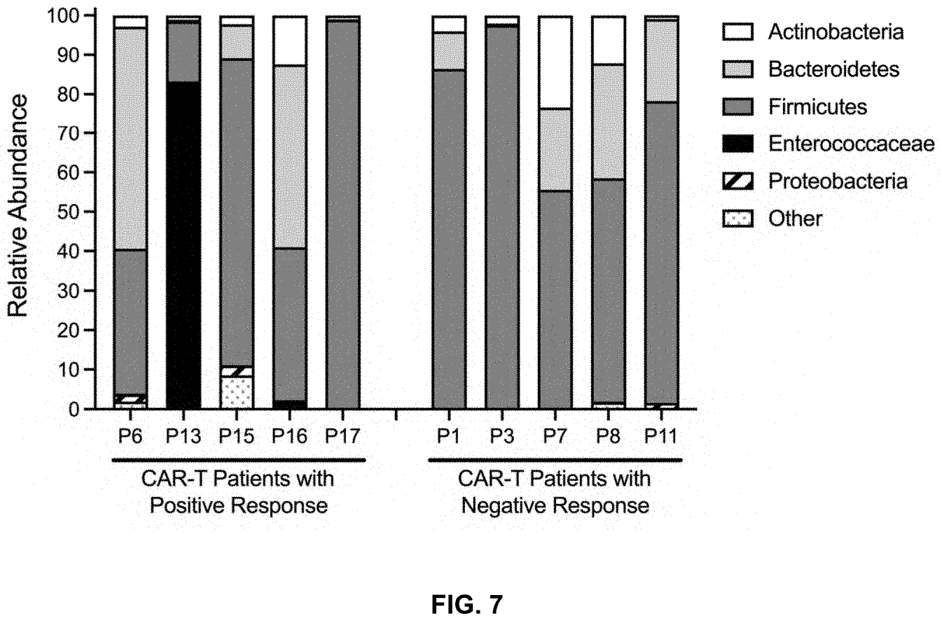

Enterococcus species have been found to be enriched in gut microbiome of patients with positive treatment response in some immunotherapies. To further investigate which bacteria are enriched in patients successfully treated with an immunotherapy, a study as indicated in FIG. 5 was performed. Briefly, pediatric patients with relapse acute B-cell lymphoblastic leukemia were treated with Kymriah (anti-CD19 CAR-T). The recipient was treated with apheresis, LD chemotherapy and CAR-T infusion as indicated. Stool and/or blood samples were obtained before and 7, 30, 60, 90, and 180 days after CAR-T cell infusion. As shown in FIGS. 6 and 7, there was a strong difference in gut microbiota composition at the day of CAR-T cell infusion among those patients who exhibited a positive clinical response (B-cell aplasia, as CD19 is a B cell marker compared to those with negative response (B-cell recovery). Differential taxonomic abundance between responder and non-responder Kymriah patients was analyzed by linear discriminate analysis coupled with effect size measurements (LEfSe) projected as a histogram (FIG. 6). All listed bacterial groups (class, order, family, genus, or species) were significantly (P<0.05, Kruskal-Wallis test) enriched for their respective phenotypic groups. (k), Kingdom. (f), Family. (g), Genus. A significant enrichment of Enterococcus spp. was noted in Kymriah CAR-T patients with positive clinical response (p=0.0053, Kruskall Wallis test).

### Example 3: Enterococcus Lysates Improve Immunotherapy Outcomes

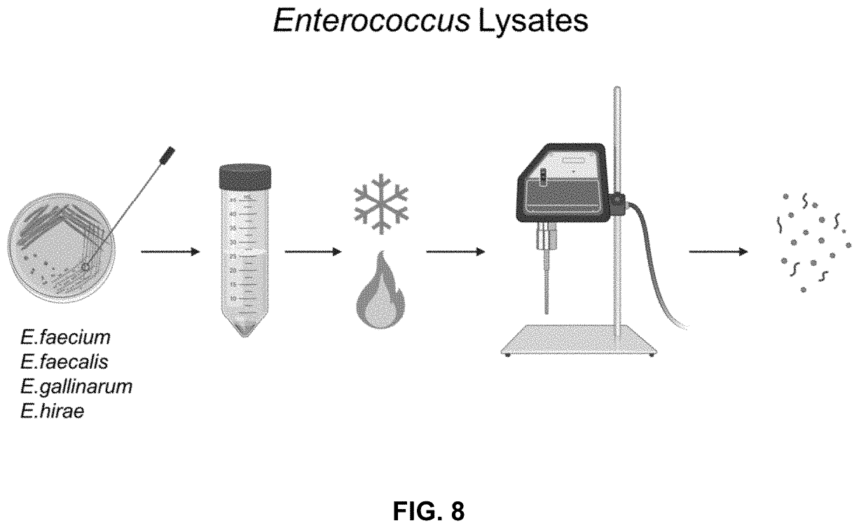

To test the role Enterococcus lysates may have on immunotherapy outcomes, Enterococcus lysates from E. faecium, E. faecalis, E. gallinarum, and E. hirae were prepared according to the following lysate production protocol, as indicated in FIG. 8: Briefly, gut microbiota were grown in the appropriate media and under appropriate conditions (anaerobic, 37° C.) overnight. 2 ml of overnight gut microbiota culture were then transferred to 100 ml of fresh media and grown under appropriate conditions until OD600=0.8-1.2. Gut microbiota were centrifuged, washed with sterile PBS three times, and resuspended in sterile PBS. Gut microbiota pellets were then flash frozen and then thawed under flowing room temperature water. Freeze/thaw process was repeated two more times, for a total of three cycles. The cell suspension was then maintained on ice. Cell suspension was then sonicated (Branson, Sonic Dismembrator, Model 1501, 40% amplitude) for 8 cycles. Each cycle consisted of 35 seconds of sonication and 35 seconds of rest. Protein concentration was determined by BCA assay at cycle 4, and then every 2 cycles until full lysis is achieved. Full lysis was defined as maximal protein concentration achieved by freeze/thaw and sonication. The cell suspension was then centrifuged (3000 g×20 min). Supernatant was removed and then filtered (0.2 um filter) into a new sterile tube. Gut microbiota lysates were then stored at 80° C. for future use

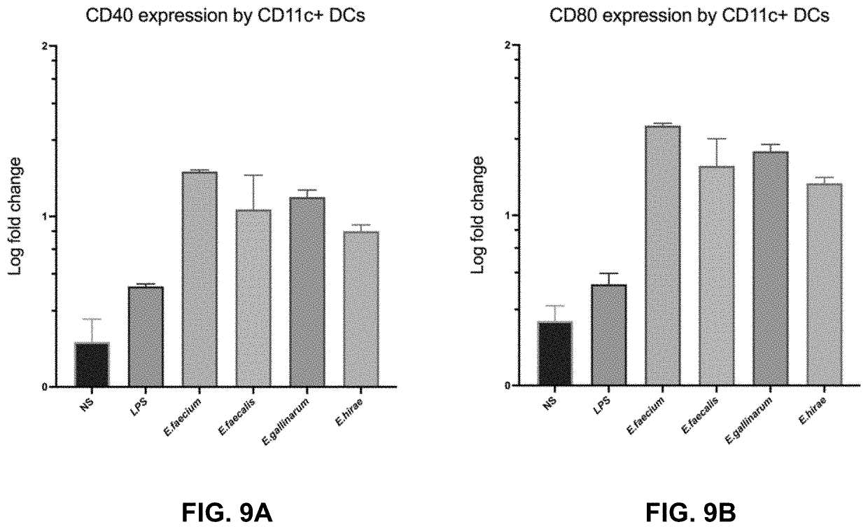

The prepared Enterococcus lysates were then applied to mouse (C57BL/6) dendritic cells (CD11c+) for 6 hours and CD40/CD80 expression was measured by flow cytometry. As shown in FIGS. 9A and 9B, application of each prepared lysate resulted in an increase in CD40+ and CD80+ expression, respectively, indicating dendritic cell activation.

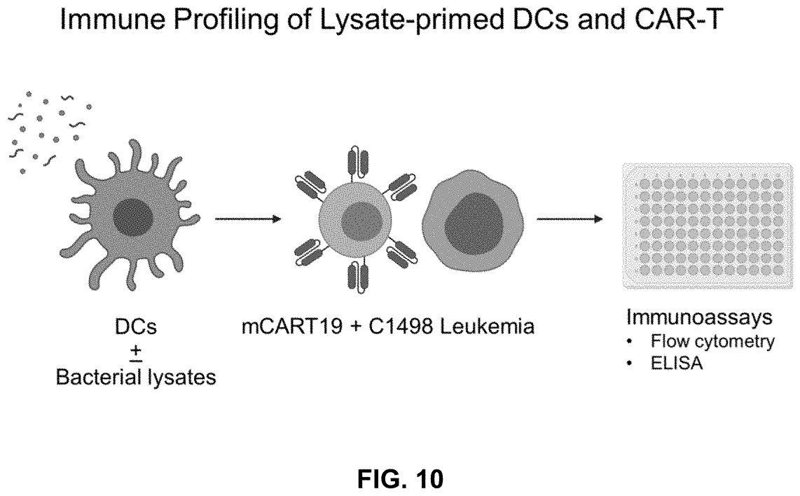

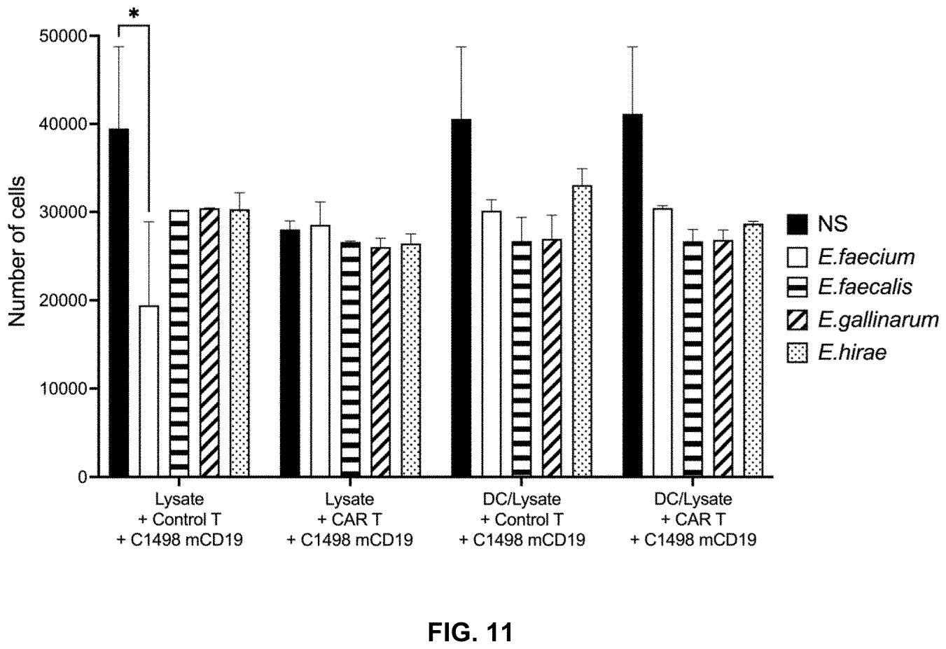

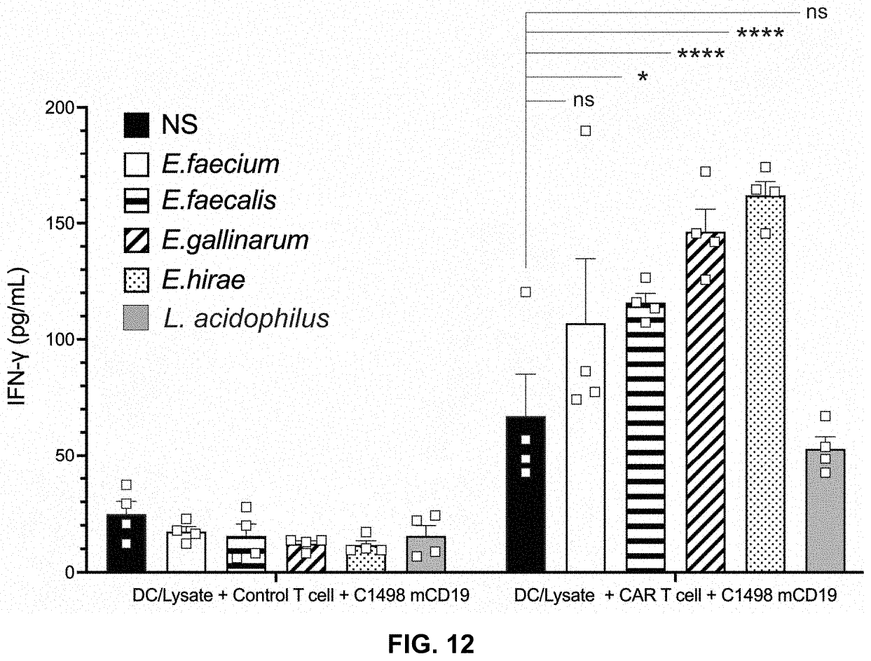

An immune-profiling experiment was then performed to test the effect of bacterial lysates on dendritic cells in the context of a CAR-T immunotherapy. The experimental scheme is shown in FIG. 10. Dendritic cells exposed to Enterococcus cell lysates as described were added to an in vitro experiment testing the effect of mCART19 CAR-T cells on C1498 leukemia cells. Flow cytometry and ELISA immunoassays were used to detect any effect the lysate exposed DC cells might have on the CAR-T cell activity. The number of viable CAR-T cells was determined by using carboxyfluorescein succinimidyl ester (CFSE) staining of T cells and flow cytometry for quantification of proliferation (FIG. 11). CAR-T cell activity was assessed by IFNy production, determined in the following protocol. 4×10⁴ dendritic cells isolated from C57BL/6 mice were co-incubated±gut microbiota lysates (final concentration 10 ug/ml, as determined by protein concentration ascertained by BCA). Dendritic cells were then co-incubated with T-cells (from C57BL/6 mice) or mouse CD19 CAR-T cells in the presence of mCD19 expressing leukemia cells (C1498) for 5-6 days. Cell culture supernatants were then assessed for IFN-γ by ELISA assay (FIG. 12). Results are from four independent experiments, with 2-3 technical replicates per experiment (One-way Anova. ns, not significant. *, p<0.05. ****, p<0.0001). Interestingly, Enterococcus-primed DCs did not directly affect CAR-T proliferation (FIG. 11) but did increase CAR-T IFN-γ production (FIG. 12).

### Example 4: Bt/Fp Lysates are Effective in Anti-PD-1 Therapy and CAR-T Therapy Alone

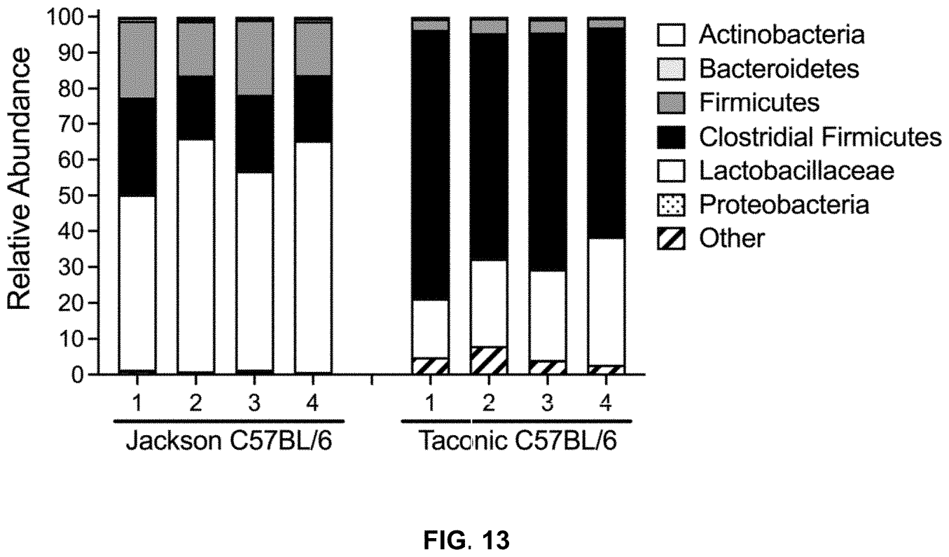

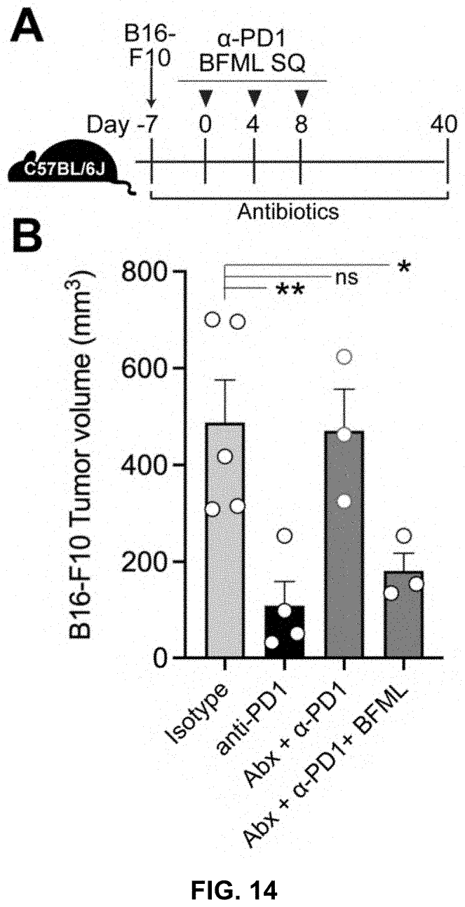

The effect of Bt/Fp lysates (prepared as described in Example 1) was tested on a B16 melanoma mouse model using anti-PD-1 alone. Leveraging the fact that C57BL6 mice from different vendors have different gut microbiomes (FIG. 13), antibiotics were used to induce an ICT hyporesponsive state in Taconic mice (but not in Jackson mice, data not shown) and then administered 200 μg anti-PD-1 and/or 3000 μg Bt/Fp microbial lysate (BFML) subcutaneously (SQ) ipsilateral, experimental schema (FIG. 14A). Consistent with earlier results, BFML administration significantly enhanced the efficacy of anti-PD-1 ICT against melanoma (FIG. 14B).

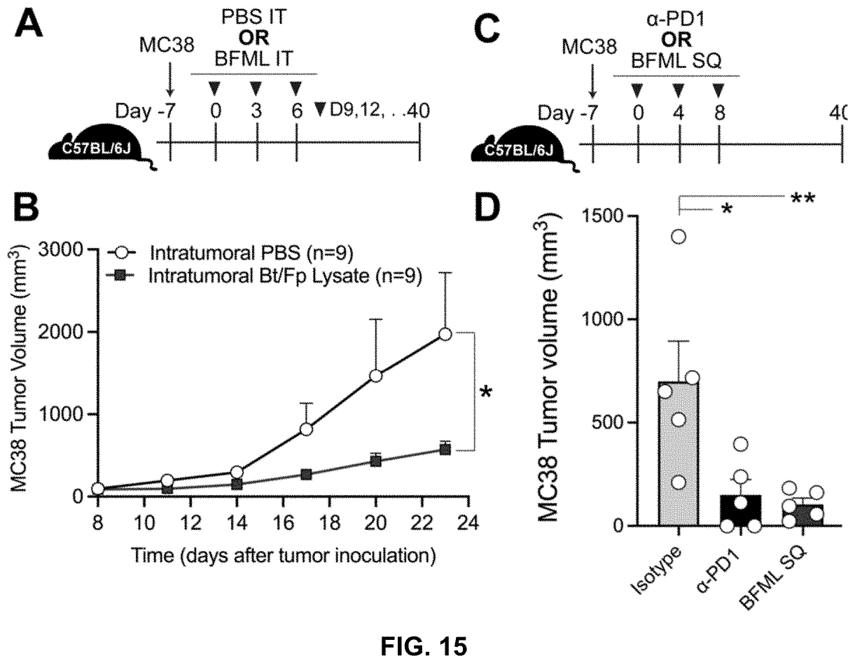

Bt/Fp Microbiota Lysates (BFML) given intratumoral (IT) and SQ enhance anti-tumor efficacy in mice with colorectal cancer (MC38). Finally, to demonstrate whether BFML is effective as a monotherapy against other cancer types, it was tested against a mouse model of MC38 colorectal cancer (1×10⁶ cells/mouse MC38 cells SQ in the right flank). In the first experiment, to test the effectiveness of intratumoral injection of BFML, mice having tumor volumes of 100 mm^{3±20} mm³ were randomized to receive either PBS IT or 100 μg BFML IT every 3 days for the duration of the experiment (FIG. 15A). Tumor growth was measured every three days starting on day 8. Injection of BFML alone markedly reduced tumor growth (FIG. 15B). Intratumoral injection of BFML alone was tested. In the second experiment, to test the effectiveness of subcutaneous administration of BFML, an ICT therapy (anti-PD-1 alone) was used as a comparator because MC38 is very sensitive to ICT. As shown in FIG. 15C, mice with MC38 tumor volumes 100 mm³±20 mm³ were randomized to receive isotype alone, anti-PD-1, BFML SQ (ipsilateral side of the tumor) alone. Surprisingly, subcutaneous injection of 3000 μg BFML reduced tumor volumes as effectively as anti-PD-1 alone, when compared to isotype controls (FIG. 15D).

### Example 5: Mechanism Behind Bt/Fp Effect on Immunotherapy

Interaction with TLRs

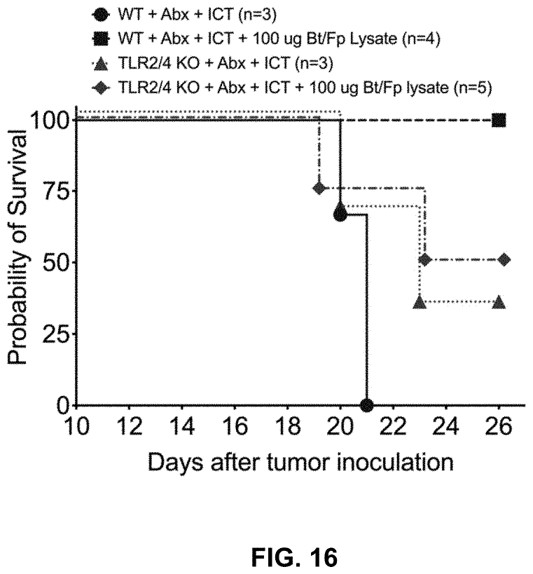

To test how Bt/Fp lysates may interact with dendritic cells to increase their activity, Bt/Fp lysates were added to WT or TLR2/4 KO mice injected with B16 tumor cells and treated with an antibiotic and an ICT (anti-PD-1 and anti-CTLA-4) treatment. Mice lacking TLR2/TLR4 showed a clear decrease in survival outcome following immunotherapy treatment with or without Bt/Fp lysates (FIG. 16).

Use of Lysates from Gram-Positive and/or Gram-Negative Bacteria

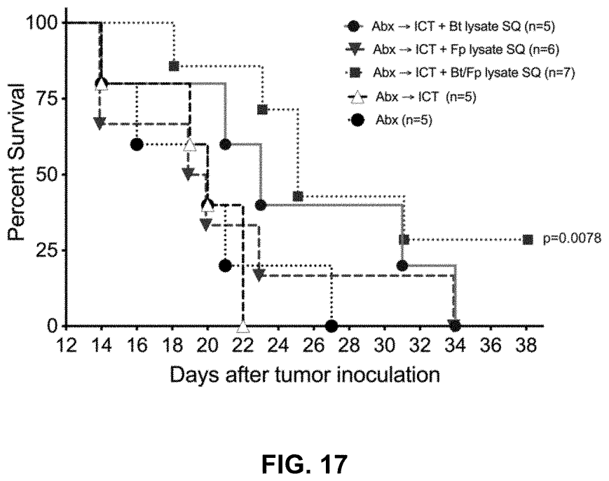

To further test survival following ICT administration alongside lysate administration, WT mice injected with tumor cells were treated with an antibiotic, ICT therapy and either (a) Bt lysate alone, Fp lysate alone or Bt and Fp lysate together. All lysates were administered subcutaneously. As shown in FIG. 17, subcutaneous administration of Bt and Fp lysates, alone or in combination, markedly improved survival of mice in a tumor model. Further, the combination of both Bt and Fp lysates showed the greatest effect on survival (FIG. 17).

### Route of Administration

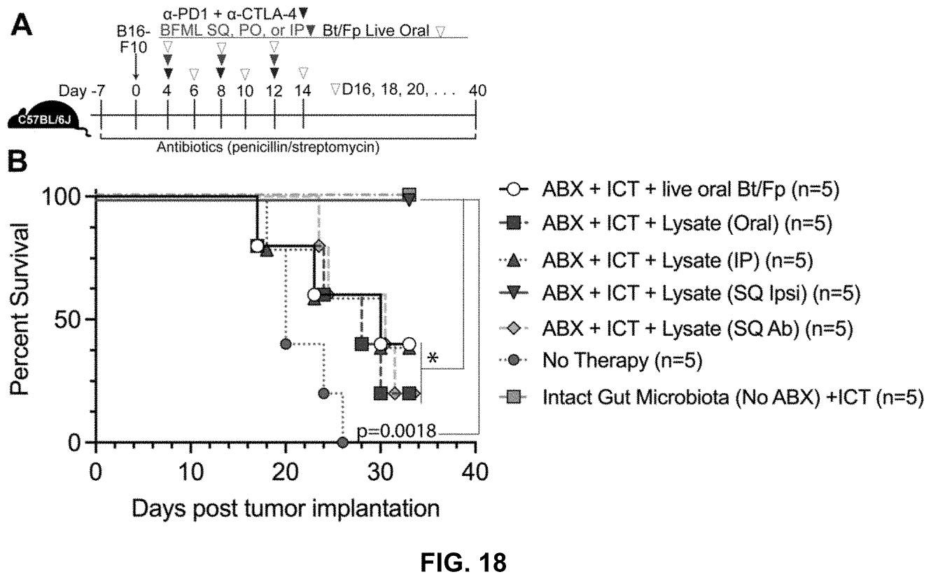

In order to better define the importance of route of administration of gut microbiota lysates, the antitumor effect of Bt/Fp microbiota lysate (BFML) administered orally, intraperitoneally (IP), or subcutaneously (SQ) was compared to administration of live oral Bt/Fp. Specifically, Jackson C57BL/6 mice were treated with antibiotics, injected with B16-F10, and treated with BFML as indicated or live Bt/Fp via oral gavage (FIG. 18A). As shown in FIG. 18B, Bt/Fp Microbiota Lysates (BFML) administered SQ (ipsilateral to tumor site) was more effective in enhancing anti-tumor (B16-F10) responses than live oral Bt/Fp.

### Effect of Gut Microbiota LPS on Dendritic Cell Activation

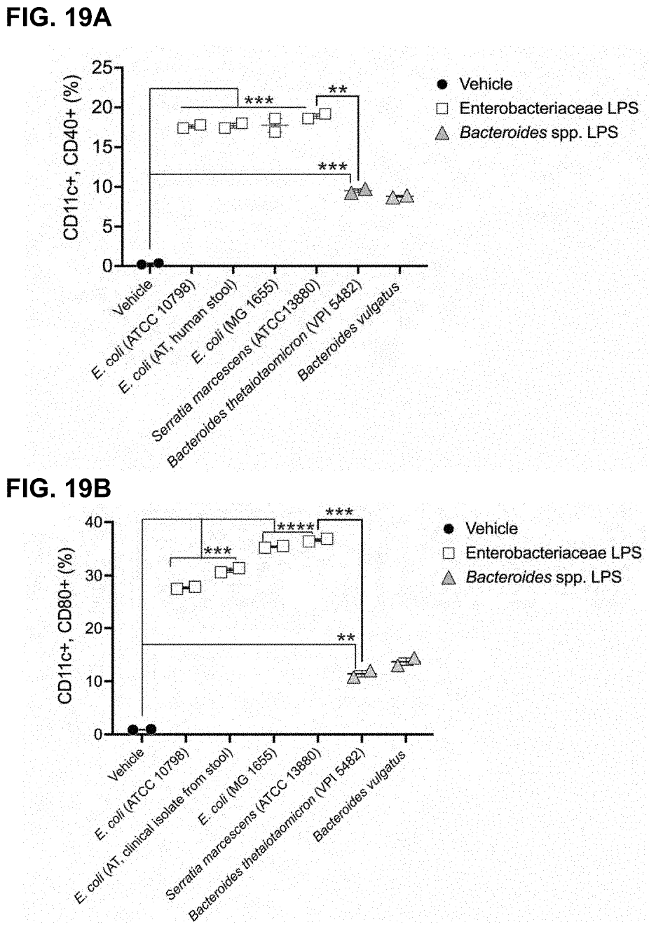

To test the role bacterial lipopolysaccharide (LPS) could have on dendritic cell activation, lysates from different species/strains of bacteria having an LPS structure similar to Enterobacteriaceae (e.g., E. coli, and Serratia marcescens) or Bacteroides spp. (e.g., B. thetaiotaomicron, or B. vulgatus) were applied to dendritic cells and levels of CD40+ or CD80+ expression was measured. As shown in FIGS. 18A and 18B, lysate from bacteria having Enterobacteriaceae LPS showed a stronger effect on dendritic activation compared to lysate from bacteria having Bacteroides spp. LPS, as measured by CD40+ expression (FIG. 19A) or CD80+ expression (FIG. 19B).

### Effect of Gut Microbiota Lipid A in Gram-Negative Bacteria on Dendritic Cell Activation

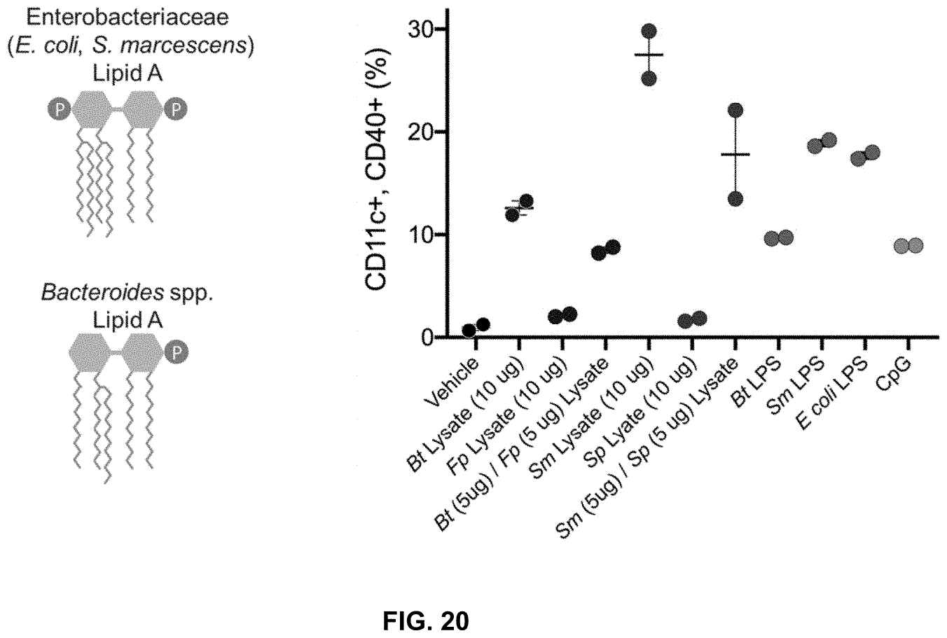

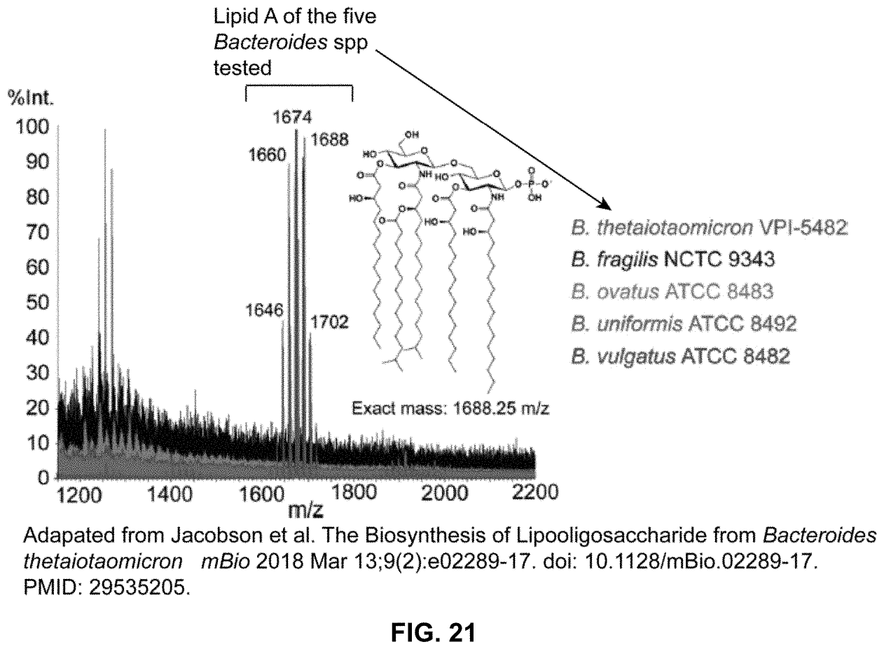

Gram-negative bacteria such as members of the Enterobacteriaceae Family (e.g., E. coli, Klebsiella spp., Serratia marcescens, etc) and Bacteroides species have different Lipid A structures, as shown in FIG. 20 (left). Specifically, Bacteroides spp have a monophosphoryl Lipid A having about 5 acyl chains (see FIG. 21 below, adapted from Jacobson et al, described below) while Enterobacteriacae species have diphosphoryl Lipid A having about 6 acyl chains. Lysates from gram negative bacteria were applied to dendritic cells. Lysates taken from bacteria having the monosphosphoryl Lipid A structure found in Bacteroides spp induced lower dendritic cell activation (measured by CD40+ expression by flow cytometry, FIG. 20, right) as compared to lysates taken from bacteria with diphosporyl lipid A (e.g., Serratia marcescens, Sm).

Previously published data has characterized differences in lipid A structure among various Gram-negative bacteria. In Jacobson et al. (“The Biosynthesis of Lipooligosaccharide from Bacteroides thetaiotaomicron” mBio. 2018 Mar. 13; 9(2):e02289-17), the Lipid A structure in various gut bacteria species was determined. In an exemplary method, a MALDI-TOF MS analysis of Lipid A isolated from five Bacteroides species was performed. Lipid A was purified from B. thetaiotaomicron VPI 5482, B. fragilis NCTC 9343, B. ovatus ATCC 8343, B. uniformis ATCC 8492, and B. vulgatus ATCC 8482 by the TRI reagent method, dissolved in 3:1 chloroform-methanol, spotted on a 5-chloro-2-mercaptobenzothiazole CMBT matrix, and analyzed on a Waters Corporation Synapt GT HTMS 32k MALDI-TOF instrument in relectron negative-ion mode. Al five have as their dominant lipid A species a cluster of peaks around 1,688 m/z corresponding to the published structure of B. thetaiotaomicron lipid A (FIG. 21). The peaks in the cluster are separated by 14 m/z (methylene group, CH₂), likely caused by heterogeneity in the number of carbons in each acyl chain of lipid A. Of note, the Bt strain used in the microbiota lysates in these examples is the same strain (VPI 5482) characterized in this publication.

### Genomic CpG Abundance in Bacteria

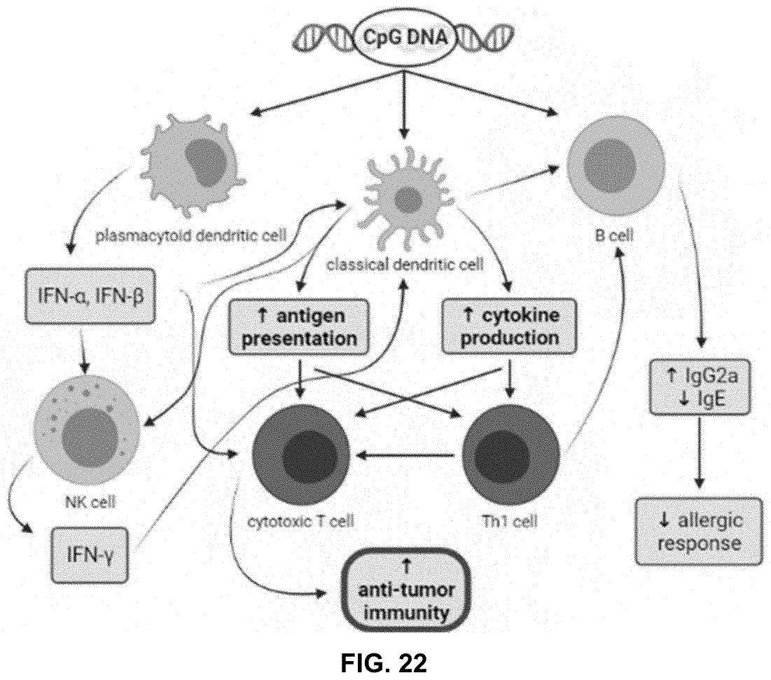

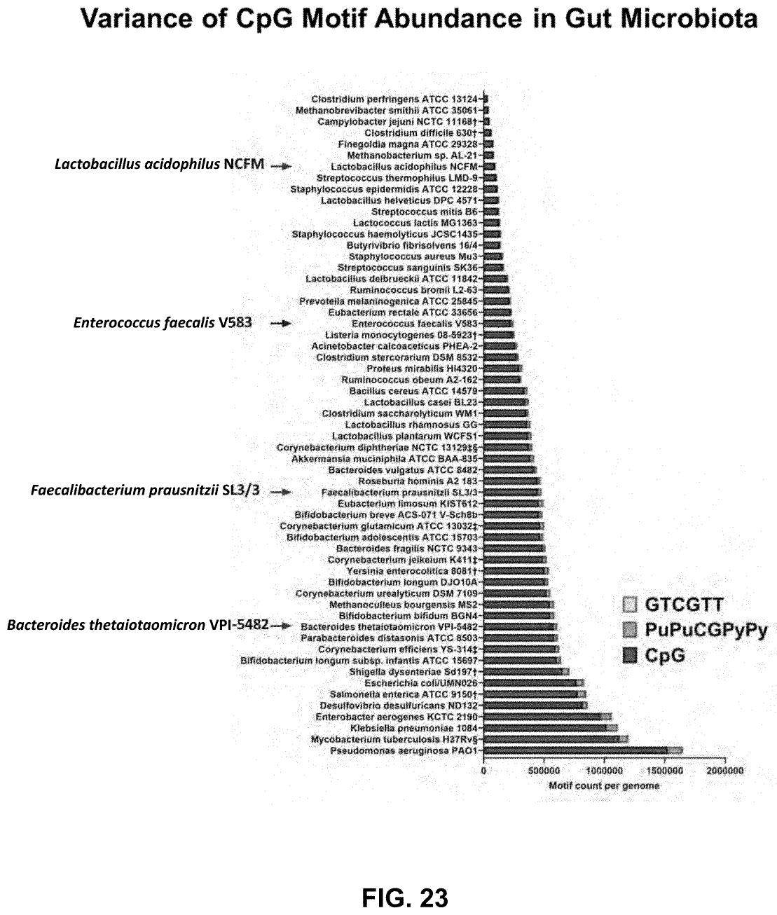

CpG DNA refers to regions of a nucleotide sequence where a cytosine is immediately followed by guanine in the 5′ to 3′ direction. It is referred to as CpG (Cytosine-phosphate-Guanine) to distinguish it from the cytosine-guanine base pair interaction. As shown in FIG. 22, CpG regions of genomic DNA have been shown to stimulate various components in the immune system. Further, as shown in FIG. 23, different bacterial species have different CpG abundances with the relative CpG abundance indicated for L. aciophilus, E. faecalis, F. prausnitzii, and B. thetaiotaomicron indicated.

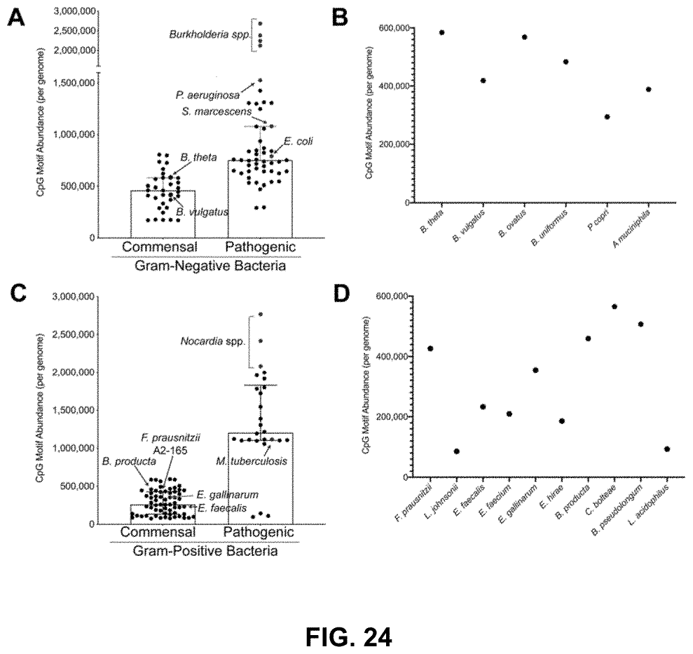

The CpG abundance of non-pathogenic vs. pathogenic Gram-negative bacteria were plotted in FIG. 24A. Non-pathogenic bacteria had a lower abundance of CpG compared to pathogenic bacteria, suggesting that candidate bacterial species for lysis should possess a calibrated level of CpG—not too high to be pathogenic, and not too low to be ignored by the immune system. Suitable Gram-negative gut microbiota candidates that had CpG motif abundance in a range similar to B. thetaiotaomicron (a representative non-pathogenic bacteria) are shown in FIG. 24B.

The CpG abundance of non-pathogenic vs. pathogenic Gram-positive bacteria were likewise plotted in FIG. 24C. As with Gram-negative bacteria, non-pathogenic bacteria had a much lower CpG abundance in their genomic DNA compared to pathogenic bacteria. Suitable Gram-positive gut microbiota candidates having a CpG motif abundance like F. prausnitii (a representative non-pathogenic bacterium) are shown in FIG. 24D.

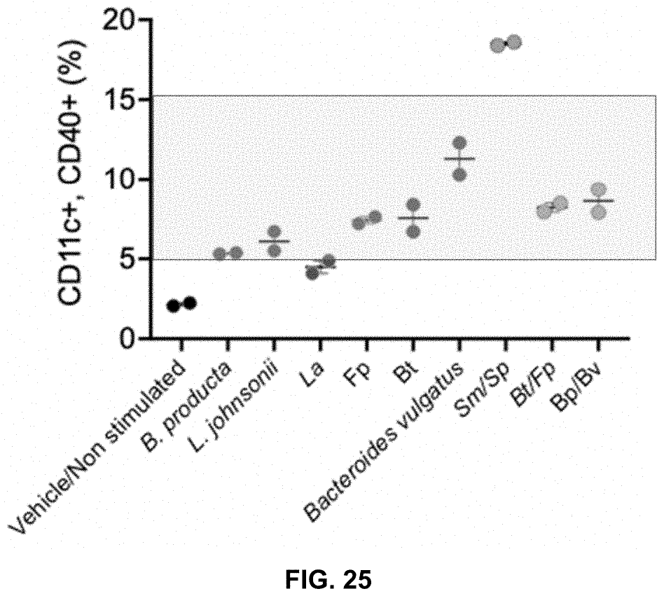

Bacteria cell lysates from bacteria in FIG. 24 were applied to dendritic cells to test the degree of activation. All bacteria having a lower amount of CpG abundance (comparable to Bt and/or Fp) were effective at activating dendritic cells (FIG. 25). Importantly, these bacteria lysates did not lead to hyperactivity, which occurred with Coley's toxin (Sm/Sp).

### Example 6: Composition of Matter of Immunoactive Components of Bacterial Cell Lysates

A series of experiments were performed to determine structural and functional properties of immunoactive components in Bt/Fp cell lysates.

Composition of Matter: Polar Vs. Non-Polar

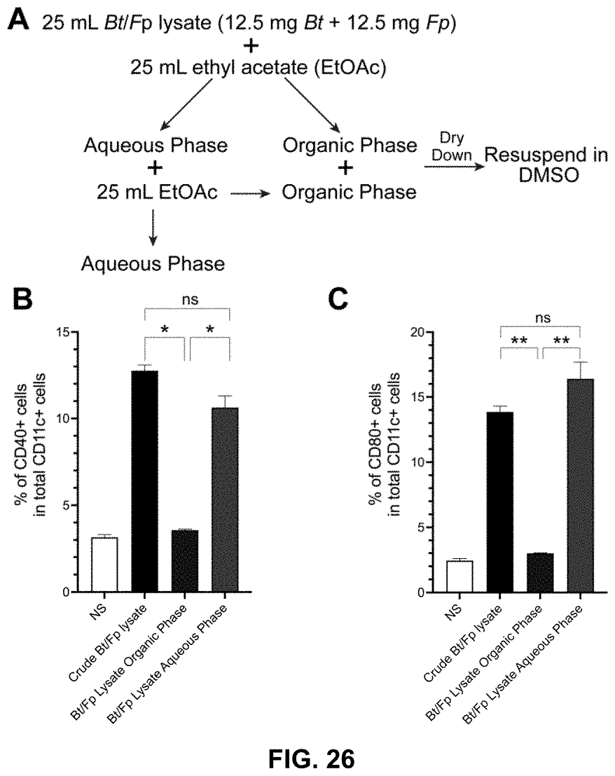

Immunoactive components of Bt/Fp lysates are polar. FIG. 26A shows an experimental scheme to separate polar and non-polar components of Bt/Fp lysates. Briefly, Bt/Fp lysates were mixed with ethyl acetate (1:1) and underwent two extractions separating polar (aqueous) and non-polar (organic) phases. Crude Bt/Fp lysates (10 μg/mL), aqueous phase (diluted 1:10), and organic phase (10 μg/mL) were co-incubated with mouse dendritic cells (CD11c+) for 6 hours. Flow cytometry for DC activation markers CD40 and CD80 was performed. The polar (aqueous) phase showed a significantly higher ability to activate DC cells compared to the nonpolar (organic) phase (FIGS. 26B and 26C, unpaired t-test. ns, not significant. *, p<0.05. **, p<0.01).

### Composition of Matter: Effect of DNAse

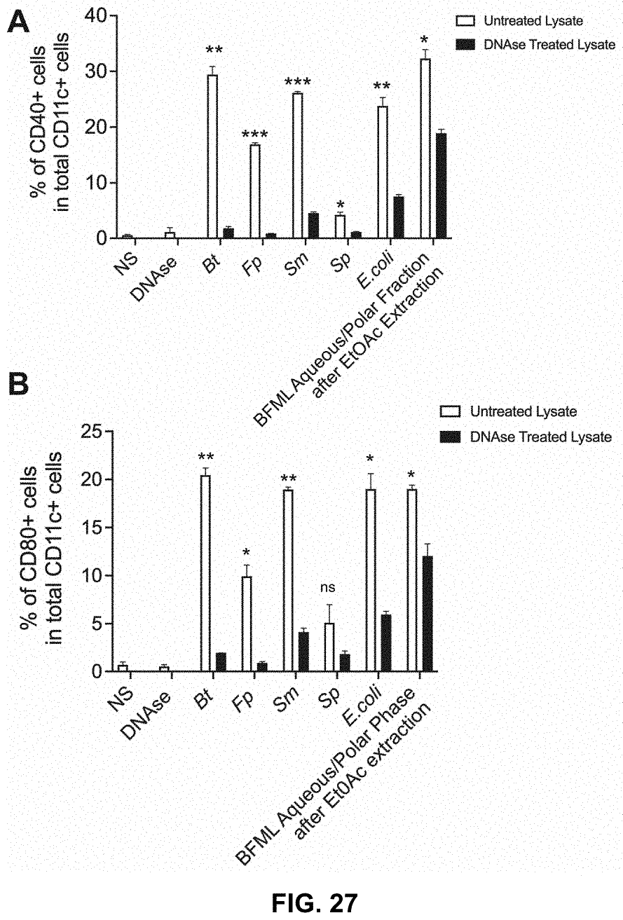

DNAse treatment of bacterial lysates decreases DC activation. Lysates from various gut microbiota were prepared as described above. DNA concentrations were ascertained by PicoGreen assay (ThermoFisher). DNAse I was added (1 unit/uL, with 1 unit of DNAse I per 1 μg gDNA) and incubated with the lysate (in a volume which contained 5 μg total of DNA per sample) for 30 minutes at 37° C. DNAse treated lysates were then co-incubated with mouse dendritic cells for 6 hours. Dendritic cell activation (CD40, CD80) was then measured by flow cytometry. DNAse treated lysate showed a significant decrease in dendritic cell activation as measured by percentage of CD40+ cells (FIG. 27A) and CD80+ cells (FIG. 27B). Unpaired t-test. ns, not significant. *, p<0.05. **, p<0.01. ***, p<0.001).

Composition of Matter: Microbiota gDNA

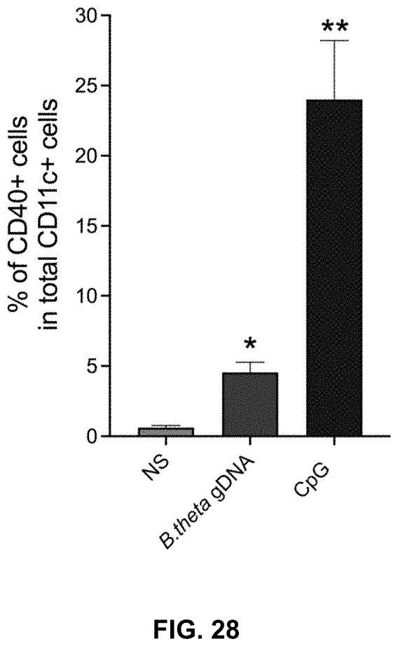

B. thetaiotaomicron gDNA activates mouse dendritic cells. Genomic DNA (gDNA) was extracted from B. thetaiotaomicron grown in vitro using standard protocols. gDNA concentrations were ascertained by PicoGreen assay (ThermoFisher). Mouse dendritic cells were co-incubated with no-stimulus PBS) control, B. thetaiotaomicron gDNA 100 μg/mL, or a positive control (CpG, 10 μg/mL) for 6 hours. Dendritic cell activation (CD40) was then measured by flow cytometry. Isolated gDNA successfully activated dendritic cells (FIG. 28, Unpaired t-test. ns, not significant. *, p<0.05. **, p<0.005.)

### Composition of Matter: Protein Denaturation

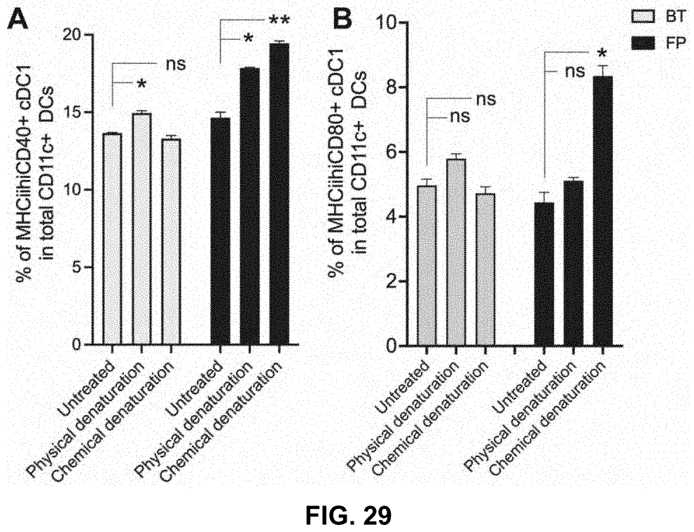

Physical and Chemical Protein Denaturation of Bt/Fp microbiota lysates does not decrease DC activation potential. For physical denaturation, gut microbiota lysates were boiled for 60 minutes. For chemical denaturation, gut microbiota lysates were treated with protease from Streptomyces griseus (a mixture of at least three proteolytic activities including an extracellular serine protease; Sigma P5147) at a final concentration of 5 mg/mL for 60 minutes at 37° C., then heat-inactivated at 80° C. for 15 minutes. Protein denatured lysates were then co-incubated with mouse dendritic cells for 6 hours. Dendritic cell activation (CD40, CD80) was then measured by flow cytometry. FIG. 29 shows that physical and chemical denaturation did not decrease the activation potential of DC cells as measured by CD40+ (FIG. 29A) or CD80+ (FIG. 29B). In fact, in some cases, protein denaturation significantly increased lysate activation of DCs. Unpaired t-test. ns, not significant. *, p<0.05. **, p<0.0.

### Gut Microbiota Lysates Agonize Distinct Mouse Pattern Recognition Receptors.

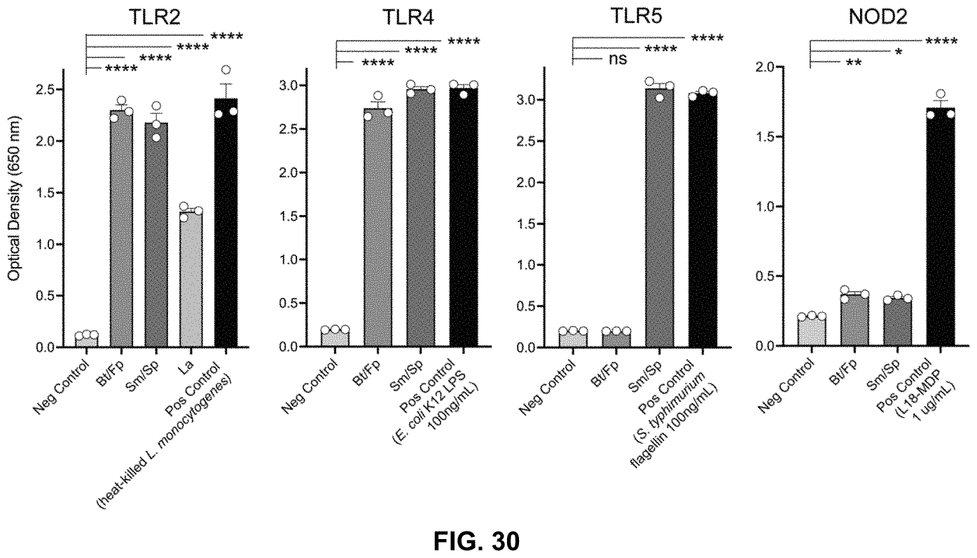

Gut microbiota lysates agonize distinct mouse pattern recognition receptors. Toll-Like Receptor (TLR), NOD-Like Receptor (NLR) and C-Type Lectin Receptor (CLR) stimulation is tested by assessing NF-κB activation in the TLR/NLR/CLR expressing HEK cell lines. The activity of the microbiota lysates was tested on eight different mouse TLRs (TLR2, 3, 4, 5, 7, 8, 9 and 13), two different mouse NLRs (NOD1 and NOD2) and one mouse CLR (Dectin-1a) as potential agonists. The secreted embryonic alkaline phosphatase (SEAP) reporter is under the control of a promoter inducible by the transcription factor NF-κB. This reporter gene allows the monitoring of signaling through the TLR/NLR/CLR, based on the activation of NF-κB. In a 96-well plate (200 μL total volume) containing the appropriate cells (50,000-75,000 cells/well), 20 μL of the lysates (10 ug/mL) or the positive control ligand was added to the wells. The media added to the wells is designed for the detection of NF-κB induced SEAP expression. After a 16-24-hour incubation the optical density (OD) was read at 650 nm on a Molecular Devices SpectraMax 340PC absorbance detector. Three technical replicates were performed for each assay. One-way Anova. *, p<0.05, **, p<0.01, ***, p<0.001, ****, p<0.0001. As shown in FIG. 30, lysates from Bt/Fp agonized TLR2, TLR4, and NOD2 but not TLR5. In contrast, Coley's Toxin (Sm/Sp) agonized TLR2, TLR4, TLR5, and NOD2.

### Summary

In general, the following observations were made following experiments in the previous examples. First, Bt/Fp lysates do not have a direct effect on CD4+ and CD8+ T cells, but they do activate dendritic cells (DCs). Second, protein denaturation (either physical by boiling or chemical by proteases) did not decrease immune activity of Bt/Fp (FIG. 29). Third, the immune-active components of Bt/Fp are polar (aqueous soluble) (FIG. 26). Fourth, bacterial species-specific differences in lipopolysaccharide (LPS) structure (e.g., lipid A) dictate the degree of DC activation. Fifth, TLR2/TLR4 are important for Bt/Fp lysate immune activity (efficacy) (FIG. 30). Finally, Bt/Fp gDNA is important for optimal DC activation (FIG. 27 and FIG. 28). As a result, it is hypothesized that Bt and Fp may work via multiple PRRs. For example, Bt lipopolysaccharide (LPS) may activate TLR4. Fp lipoteichoic acid (LTA) may activate TLR2. Bt and/or Fp lipids may activate TLR4. Bt and/or Fp genomic DNA may stimulate TLR9. Bt and Fp cell wall components (peptidoglycan) may activate NOD2 (FIG. 9)

### Example 7—Experiment to Test Effect of B. vulgatus and B. productus on Immunotherapy

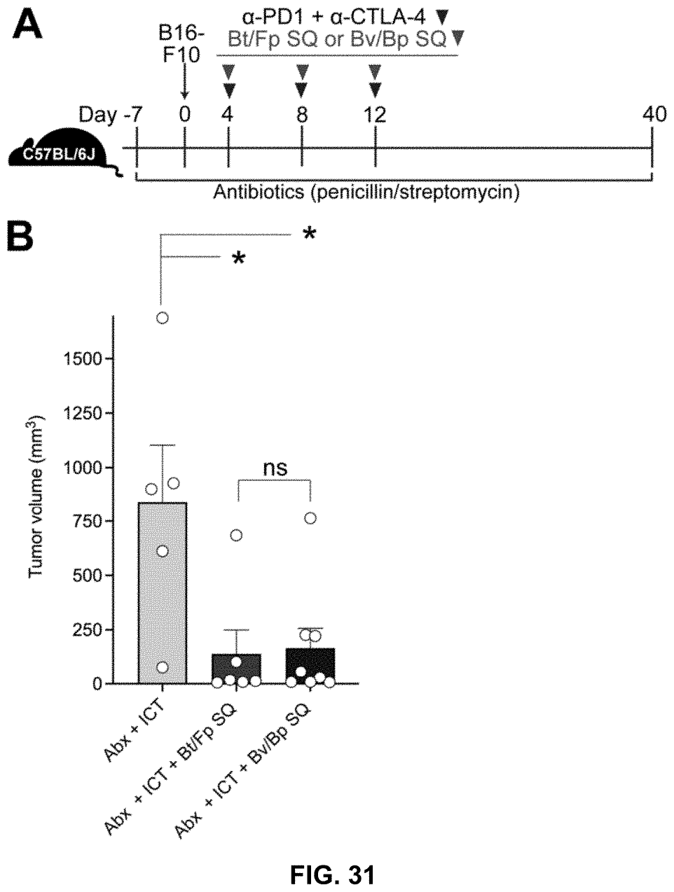

The data in Examples 1 to 6 suggest that other combinations of Gram-positive and Gram-negative bacteria (other than Bt/Fp) should improve immunotherapy outcomes. To test this, a protocol diagrammed in FIG. 31A was performed. C57/BL6 mice were administered antibiotics for seven days prior to injection with B16-F10 cancer cells. ICT (200 μg anti-PD-1 antibody and 200 μg anti-CTLA-4 antibody, administered intraperitoneally) was administered from days 4 to 12 after tumor injection. Cell lysate prepared from gut Gram-negative B. vulgatus and gut Gram-positive B. producta were administered on days 4, 8 and 12. Tumors were measured daily. Three groups were tested: Group 1: Antibiotic+ICT (n=5) (negative control, hyporesponseive state); Group 2: Antibiotic+ICT+B. theta/F. prausnitzii, subcutaneously (100/100 μg) ipsilateral to the tumor injection (n=6); and Group 3: Antibiotic+ICT+B. vulgatus/B. producta subcutaneously (100 μg/100 ug) ipsilateral to the tumor injection (n=8). Both Bt/Fp and By/Bp lysate groups had significantly small tumor volumes (D+22 after tumor inoculation) compared to the Abx+ICT group (FIG. 31B). Of note, By/Bp had an equivalent effect to Bt/Fp as measured by absolute tumor volume relative to the control group.

All publications, patents and patent applications mentioned in this specification are herein incorporated in their entirety by reference into the specification, to the same extent as if each individual publication, patent or patent application was specifically and individually indicated to be incorporated herein by reference. In addition, citation, or identification of any reference in this application shall not be construed as an admission that such reference is available as prior art to the present invention. To the extent that section headings are used, they should not be construed as necessarily limiting.

1. (canceled)

2. A pharmaceutical composition, the composition comprising: one or more components from one or more bacterial lysates from one or more species of a Gram-positive bacterial cell; one or more components from one or more bacterial lysates from one or more species of a Gram-negative bacterial cell; and at least one pharmaceutically acceptable carrier and/or excipient.

3. The pharmaceutical composition according to claim 2, wherein the one or more species of a Gram-positive bacterial cell is F. prausnitzii, F. johnsonii, E. faecalis, Enterococcus sp., E. faecium, E. gallinarum, E. hirae, B. producta, C. bolteae, B. pseudolongum, L. acidophilus, or any combination thereof and the Gram-negative bacterial lysate comprises lysate from B. thetaiotaomicron, B. vulgatus, B. ovatus, B. uniformus, P. copri, or A. muciniphila.

4. The pharmaceutical composition according to claim 3, wherein the one or more species of a Gram-negative bacterial cell is B. thetaiotaomicron, B. vulgatus, or any combination thereof.

5. The pharmaceutical composition according to claim 3, wherein the one or more species of a Gram-positive bacterial cell is E. faecium, E. faecalis, E. gallinarum, E. hirae, or any combination thereof.

6. A pharmaceutical composition, the composition comprising one or more bacterial lysates from one or more species of bacteria having genomic DNA with a CpG abundance substantially similar to a CpG abundance in genomic DNA of a gut bacterium and a pharmaceutically acceptable carrier and/or excipient.

7. The composition according to claim 6, wherein one or more species of bacteria having genomic DNA with a CpG abundance substantially similar to a CpG abundance in genomic DNA of a gut bacterium is F. prausnitzi, B. thetaiotaomicron, B. producta, B. vulgatus, or any combination thereof.

8. The composition according to claim 6, wherein the one or more species of gut bacterium is F. prausnitzi, B. thetaiotaomicron, B. producta, B. vulgatus, or any combination thereof.

9. A pharmaceutical composition, the composition comprising one or more bacterial lysate from a species of bacteria that comprises a Lipid A structure substantially similar to a Lipid A in B. thetaiotaomicron and at least one pharmaceutically acceptable carrier and/or excipient.

10. The pharmaceutical composition according to claim 9, wherein the Lipid A structure comprises a monophosphoryl Lipid A comprising 5-6 acyl chains.

11. The pharmaceutical composition of claim 2, wherein the one or more species of Gram-negative bacterial cell comprises a monophosphoryl Lipid A comprising 5-6 acyl chains such that Gram-negative bacterial lysate comprises the same, and wherein the one or more Gram-positive bacterial lysates and/or the one or more Gram-negative bacterial lysates comprise genomic DNA with a CpG abundance substantially similar to that of a CpG abundance in genomic DNA of a gut bacterium.

12. The pharmaceutical composition of claim 11, wherein the one or more species of Gram-positive bacterial cells comprise lipoteichoic acid (LTA) having a structure substantially similar to the LTA found in F. prausnitzii.

13. The pharmaceutical composition of claim 11, wherein one or more species of Gram-positive bacterial cell is F. prausnitzii.

14. The pharmaceutical composition of claim 13, wherein the one or more Gram-positive bacterial lysates and one or more Gram-negative bacterial lysates collectively contain ligands that are capable of binding to toll-like receptor 2, toll-like receptor 4, and NOD2 on a target cell, and such binding being sufficient to activate a cellular response in such target cell.

15. The pharmaceutical of claim 1, wherein the composition is formulated as a liquid formulation and the pharmaceutically acceptable carrier and/or excipient comprises a phosphate buffered saline solution.

16. The pharmaceutical composition of claim 15, wherein the liquid formulation comprises a pH of from about 6.8 to 7.5.

17-18. (canceled)

19. A method for the treatment of cancer in a subject in need thereof, the method comprising: (a) administering a therapeutically effective amount of the pharmaceutical composition of claim 2 to the subject.

20. The method of claim 19, wherein the method further comprises (b) administering a therapeutically effective amount of a cancer treatment to the subject.

21. The method of claim 20, wherein steps (a) and (b) are administered at least partially simultaneously.

22. The method according to claim 19, wherein step (a) comprises orally administering the pharmaceutical composition to the subject.

23. The method according to claim 19, wherein step (a) comprises parenterally administering the pharmaceutical composition to the subject.

24. The method according to claim 23, wherein the parenteral administration of the pharmaceutical composition in step (a) is intravenous, intraperitoneal, intramuscular, intrathecal, or subcutaneous, or any combination thereof.

25. (canceled)

26. The method according to claim 24, wherein step (a) comprises subcutaneously administering the pharmaceutical composition ipsilaterally to a tumor in the subject.

27. The method according to claim 24 wherein step (a) comprises subcutaneously administering the pharmaceutical composition contralaterally to a tumor in the subject.

28-29. (canceled)

30. The method according to claim 24 wherein step (a) comprises administering the pharmaceutical composition intravenously, and administering the pharmaceutical composition or a second pharmaceutical composition comprising: one or more components from one or more bacterial lysates from one or more species of a Gram-positive bacterial cell; one or more components from one or more bacterial lysates from one or more species of a Gram-negative bacterial cell; and at least one pharmaceutically acceptable carrier and/or excipient subcutaneously close to a draining lymph node of a metastasis.

31. The method of claim 20, wherein the cancer treatment is an immunotherapy treatment.

32. The method according to claim 31, wherein the cancer immunotherapy treatment comprises administering to the subject an immune checkpoint inhibitor (ICT), modified immune cells, a bispecific antibody, or any combination thereof.

33. The method according to claim 32, wherein the cancer immunotherapy treatment comprises administering an immune checkpoint inhibitor (ICT) and the immune checkpoint inhibitor (ICT) comprises an anti-PD-1 therapy, an anti-PD-L1 therapy, an anti-CTLA-4 therapy or any combination thereof.

34. The method according to claim 33, wherein the anti-PD-1 therapy comprises pembrolizumab, nivolumab, cemiplimab, spartalizumab, sintilimab, tislelizumab, toripalimab, dostalimab or combinations thereof; the anti-PD-L1 therapy comprises atezolizumab, avelumab, durvalumab KN035, AUNP12, or combinations thereof, and/or the anti-CTLA-4 therapy comprises ipilimumab.

35. The method according to claim 33, wherein the cancer immunotherapy treatment comprises administering (a) an anti-PD-L1 or anti-PD-1 therapy; and (b) an anti-CLTA-4 therapy.

36. The method according to claim 31, wherein the pharmaceutical composition comprises one or more bacterial lysates from F. prausnitzii, B. thetaiotaomicron, or any combination thereof.

37. The method according to claim 32, wherein the cancer immunotherapy treatment comprises administering modified immune cells to the subject and the modified immune cell comprises a modified natural killer (NK) cell, a modified dendritic cell (DC), a CAR-T cell or any combination thereof.

38. (canceled)

39. The method according to claim 19, wherein the cancer is selected from the group consisting of squamous cell head and neck cancer, colon cancer, colorectal cancer, Acute myeloid leukemia (AML), Chronic myeloid leukemia (CML) Acute lymphoblastic leukemia (ALL), Merkel cell carcinoma, cutaneous squamous cell carcinoma, hepatocellular carcinoma, advanced renal cell carcinoma, microsatellite instability-high (MSI-H) or mismatch repair deficient (dMMR) cancers, cervical cancer, small cell lung cancer, non-small cell lung cancer, triple-negative breast cancer, gastric and gastroesophageal junction (GEJ) carcinoma, classical Hodgkin lymphoma, primary mediastinal B-cell lymphoma (PMBCL), and locally advanced or metastatic urothelial cancer.

40. The method according to claim 39 wherein the cancer is colon cancer or colorectal cancer, melanoma, metastatic melanoma, acute B-cell lymphoblastic leukemia, or non-small cell lung cancer.

41-44. (canceled)

45. The method according to claim 19, wherein step (a) comprises administering from about 0.0005 mg/kg to about 10 mg/kg of the pharmaceutical composition to the subject.

46. The method or use according to claim 45, wherein step (a) comprises administering from about 0.3 to 9.8 mg/kg of the pharmaceutical composition to the subject.

47. A kit for use in the treatment of cancer in a subject in need thereof, comprising:
i) one or more gut bacterial lysates in a composition formulated for oral or parenteral administration, wherein the one or more gut bacterial lysates in a composition formulated for oral or parenteral administration is a pharmaceutical composition according to claim 2; and
ii) a cancer immunotherapy treatment comprising:
(a) one or more compositions suitable for use in immune checkpoint inhibitor therapy (ICT);
(b) one or more compositions suitable for immune cell transfer therapy;
or
(c) a bispecific antibody.

48-50. (canceled)
